(19) United States
(12) Patent Application Publication
(10) Pub. No.: US 2015/0272900 A1
(43) Pub. Date: Oct. 1, 2015

(54) Layer-By-Layer Approach to Co-Deliver DNA and siRNA via AuNPs: A Potential Platform for Modifying Release Kinetics

(71) Applicant: THE JOHNS HOPKINS UNIVERSITY, Baltimore, MD (US)

(72) Inventors: Jordan J. Green, Nottingham, MD (US); Corey J. Bishop, Baltimore, MD (US)

(21) Appl. No.: 14/438,348

(22) PCT Filed: Oct. 28, 2013
(86) PCT No.: PCT/US13/67099

§ 371 Date: Apr. 24, 2015

Related U.S. Application Data
(60) Provisional application No. 61/719,088, filed on Oct. 26, 2012.

(51) Int. Cl. A61K 9/51; A61K 31/7088; A61K 31/713

(52) U.S. Cl. CPC A61K 9/5146; A61K 31/713; A61K 31/7088; A61K 9/5115

(57) ABSTRACT

A layer-by-layer (LbL) system, which alternately ionically complexes anionic AuNPs to two unique cationic polymers (disuifide-reducible and hydrolytically degradable) and two anionic nucleic acids, is disclosed.

### FEDERALLY SPONSORED RESEARCH OR DEVELOPMENT

This invention was made in part with United States Government support under DGE-0707427 awarded by the National Science Foundation (NSF) and R21CA 152473 awarded by the National Institutes of Health (NIH). The U.S. Government has certain rights in the invention.

### BACKGROUND

A host of diseases caused by genetic disorders exist that could be substantially mitigated or cured by gene therapy. No FDA-approved gene therapies are available to date, however, due to a lack of safety and efficacy. Viral vectors have been associated with immune complications and cancer although they have excellent transfection capabilities, whereas polymeric vectors are generally safer than viral vectors, but lack efficiency. Improved nucleic acid vectors are needed for clinical translation. Inorganic gold nanoparticles (AuNP) are a promising gene delivery vector as they are monodisperse, biocompatible, readily surface modifiable, and have unique optical properties. Sunshine, et al. Therap. Delivery, 2011, 2(4), 493-521.

### SUMMARY

In some aspects, the presently disclosed subject matter provides a composite comprising a polymeric network or gel and an inorganic nanoparticle, wherein the inorganic nanoparticle can generate heat upon external stimulation.

In other aspects, the presently disclosed subject matter provides a composite comprising a core inorganic nanoparticle and one or more layers or coatings of a polyelectrolyte. In some aspects, the one or more layers or coatings of a polyelectrolyte comprise one or more layers or coatings of materials that alternate in charge between positive and negative. In some aspects, the one or more layers or coatings comprise a charged biological molecule.

In both embodiments of the presently disclosed composites, the polymeric network, gel, or polyelectrolyte can comprise a degradable polymer. In certain aspects, the polymeric network or gel comprises a compound synthesized by the following method, including one or more of the following monomers and combinations thereof:

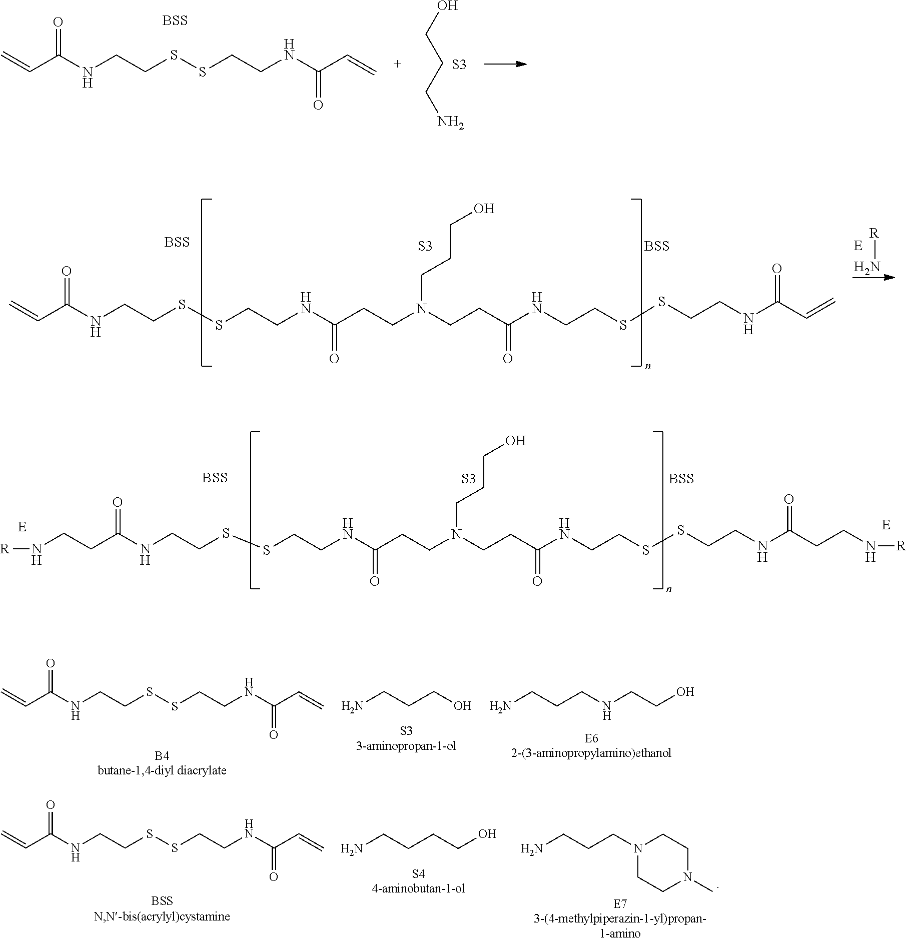

In other aspects, the polymeric network, gel, or polyelectrolyte comprises one or more backbones and side chains selected from the following monomers:

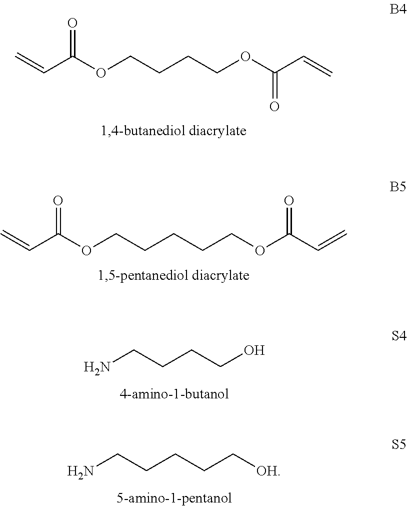

In certain aspects, the inorganic nanoparticle comprises a gold nanoparticle. In some aspects, the gold nanoparticle can be activated when exposed to a particular wavelength of light. In other aspects, the inorganic nanoparticle comprises a magnetically-activated nanoparticle.

In further aspects, the composite further comprises a cargo. In certain aspects, the cargo is selected from the group consisting of a therapeutic agent, a biosensor, and a biological molecule. In more particular aspects, the therapeutic agent is selected from the group consisting of a gene, DNA, RNA, siRNA, miRNA, isRNA, agRNA, smRNA, a nucleic acid, a peptide, a protein, a chemotherapeutic agent, a hydrophobic drug, a small molecule drug, and combinations thereof. In certain aspects, the therapeutic agent can be released from the composite in response to a change in temperature of the composite, e.g., in response to a thermal stimulus.

In yet further aspects, the presently disclosed matter provides an implant or biosensor comprising the presently disclosed composites. In some aspects, the implant is suitable for on-demand or extended release delivery of a therapeutic agent to a subject.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 is a TEM of monodisperse gold nanoparticles (AuNPs) 20 nm in size;

FIG. 2 shows the population percentages batch to batch are similar for given diameters which show consistency in the synthesis method;

FIG. 3 is the surface plasmon resonance (SPR) wavelength of pure AuNP solution at approximately 5e11 particles per mL;

FIG. 4 is the SPR wavelength of AuNPs vs TEM diameter,

FIG. 5 shows DLS vs TEM of naked AuNPs;

FIG. 6 shows NanoSight vs TEM of naked AuNPs;

FIG. 7 shows the zeta potential (ZP) of naked AuNPs vs TEM;

FIG. 8 shows the ZP vs [AuNP];

FIG. 9 illustrates AuNPs' SPR red-shifting due to aggregation because of the decreasing pH;

FIG. 10 is a schematic for synthesizing the presently disclosed monomers and polymers:

FIG. 11 shows the zeta potential versus polymer concentration as used when layering the first layer of polymer;

FIG. 12 shows the hydrodynamic radius of AuNPs after the first layer of polymer at various polymer concentrations;

FIG. 13A shows AuNP: 58.3 μL, 1E11 particles/mL in water; BSS-S3-E7: 41.7 μL, 5 mg/mL; pEGFP:41.7 μL, 0.5 mg/mL; siRNA: 41.7 μL, 4 μM; B4-S4-E6: 41.7 μL, 0 mg/mL, 0.5 mg/mL, 2 mg/mL, or 5 mg/mL; after each layer the nanoparticles were centrifuged at 1.5 kref for 10 min;

FIG. 13B is a schematic of a representative layer-by-layer nanoparticle;

FIG. 14 shows the zeta potential of AuNP/polymer/DNA ionic complexes at various DNA concentrations;

FIG. 15 shows the reversal of zeta potential after each successive layer;

FIG. 16 shows the diameter of AuNPs after each successive layering;

FIG. 17 is a TEM of completely layered AuNPs showing aggregation;

FIGS. 18A-18I show Accuri flow cytometry FL1 (EGFP) vs FL2 (Cy3; tagged DNA and siRNA) dot plots depicting uptake of nanoparticles 4 hours post transfection; All plots had >1000 cell counts; A: Untreated; B: Lipofectamine 2000; C: 446 1.2 polyplex; D: LbL ending in poly(ethylene amine) (25 kDa; 2 mg/mL); E-I: PBAE as last layer at 0 mg/mL, 0.5 mg/mL, 2 mg/mL, and 5 mg/mL, respectively;

FIG. 19 shows heating curves of spherical (20 nm) and branched Au nanoparticles (60 nm). Laser conditions: 690 nm, 0.04 density filter, approximately 140 mW;

FIG. 20 shows synthesized B4S4, PEGDA 700 (10:20) with ratios of nanoparticles. Branched Au nanoparticles (upper) and spherical Au nanoparticles (lower);

FIG. 21 shows UV-Vis absorbance graphs of gels synthesized with Au nanoparticles;

FIG. 22 are dark field micrographs, which show homogeneous distribution of Au nanoparticles in B4S4 PEGDA 700 gel (right) versus empty gel (left);

FIGS. 23A-23C show heating curves of branched (60 nm) Au nanoparticles. Laser conditions: 690 nm, 0.04 density filter, approximately 140 mW:

FIG. 24A-24C show heating curves of spherical (20 nm) Au nanoparticles. Laser conditions (left, middle): 690 nm, 0.04 density filter, approximately 140 mW. Laser conditions (right): 690 nm, 0.04 density filter, approximately 160 mW;

FIGS. 25A-25B are concentration vs. absorbance graphs obtained using a Tecan Plate Reader indicates a linear relationship that can be used, for example, for drug release and retention experiments;

FIGS. 26A-26B show (A) disulfide-reducible poly(amidoamine), BSS-S3-E7 (Lin et al., 2007) and (B) hydrolytically degradable poly((3-amino ester), B4-S4-E7 (Bhise et al., 2010);

FIG. 27 shows a schematic of the process by which M-AuNPs are coated by polymer and nucleic acid (NA) layers;

FIG. 28 shows a TEM of monodisperse, 15-nm citrate-stabilized AuNPs; 200-nm scale bar,

FIG. 29 shows the transfection efficacy and relative metabolic activity of various formulations. P is polyethylenimine (PEI), D is DNA, 447 and SS37 are the B4-S4-E7 and BSS-S3-E7 polymers, respectively. LbL is MAuNP-P-D-SS37-siRNA-447;

FIG. 30 shows knockdown in time of the LbL, Lipofectamine and 447 formulations;

FIG. 31 shows dsRed expression at day 2 (6A-6D): (6A) LbL 1.5× dose, (6B) LbL, (6C) Lipofectamine, (6D) 447; eGFP knockdown at day 9 (6E-6H): (6E) LbL eGFP siRNA, (6F) LbL scr-siRNA, (60) Lipofectamine eGFP siRNA, (6H) Lipofectamine scr-siRNA; and

FIG. 32 shows the reversal of zeta potential after each successive layer (left) and diameter of each of the layers (right) after two washings using the LbL formulation.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Nanocomposites of Gold and Polymers

The challenge of delivering biological cargos, such as peptides, nucleic acids, imaging agents, and chemotherapies in a controlled way over time remains despite extensive research in this area. The presently disclosed subject matter, in some embodiments, allows “on-demand” delivery of these cargos.

Generally, the presently disclosed subject matter provides combinations and formulations of polymer-based systems for release of biological agents. More particularly, the presently disclosed subject matter provides a composite formed of a polymeric network or gel and an inorganic nanoparticle type that generates heat upon external stimulation. In many embodiments, the polymers are degradable. In some embodiments, the polymers have a specific structure. In one embodiment, gold nanoparticles are included that are active when exposed to a certain wavelength of light. In another embodiment, magnetically-active nanoparticles are provided. In some embodiments, this change in temperature causes release of agents including, but not limited to, chemotherapies, biosensors, biological molecules, and the like. In some embodiments, the presently disclosed system can be implanted under the skin of a subject for on-demand drug delivery to the patient.

In other embodiments, a composite formed by a core inorganic nanoparticle and coatings or layers of polyelectrolytes is provided. In some embodiments, the polymers have a specific structure. In one embodiment, gold nanoparticles comprise the core particle. In many embodiments, the coatings will alternate in charge between positive and negative. In some embodiments, the coatings will include charged biological agents or drugs including, but not limited to, peptides or nucleic acids. In some embodiments the layers are degradable. In some embodiments, the layers respond to external triggers, like the composites disclosed immediately hereinabove. In many embodiments, the presently disclosed composites are useful as sensors and/or to release agents/drugs over time. In some embodiments, the particles have a dimension of about 20 nm to about 100 nm; about 100 nm to about 500 nm; about 500 nm to about 1000 nm; about 1 micron to about 10 microns; and from about 10 to about 30 microns.

### II. Nano-Gold/Degradable Polymer Hybrid Nanoparticles for Co-Delivery of DNA and siRNA

In some embodiments, the presently disclosed subject matter demonstrates that siRNA and DNA can be simultaneously ionically complexed to gold nanoparticles (AuNPs) for co-delivery using two cationic polymers having unique degradable mechanisms. One such polymer, BSS-S3-E7, is a disulfide-containing-poly(amidoamine), which can be reduced and degraded upon uptake into the cell with increased glutathione levels. In other embodiments, B4-S4-E6 is a poly(β-aminoester), which is degraded by hydrolysis. The two uniquely degrading polymers allow the release kinetics of DNA and siRNA to be tuned by varying the order and the number of the layers of the polymers. Further, varying the disulfide density within the poly(amido amine) polymer will allow further control over the release kinetics of the DNA and siRNA.

The LbL layer ending in PBAE appears to be superior to PEI and Lipofectamine 2000 and appears to be comparable to PBAE polyplexes in endosomal uptake in the glioblastoma cell line used according to the flow cytometry uptake data. Without the PBAE on the outer coating of the particles, the LbL AuNPs are not uptaken into the cells. Increasing LbL PBAE concentrations increases uptake which will likely lead to enhanced transfection.

Accordingly, the presently disclosed subject matter provides a theranostic technology that can deliver combinations of genetic therapies along with an agent for imaging and potential photothermal therapy.

In some embodiments, the presently disclosed subject matter provides a nanoparticle comprising: a nanoparticle core; a first layer comprising a first cationic polymer, a second layer comprising a first anionic nucleic acid; a third layer comprising a second cationic polymer, wherein the first and the second cationic polymer can be the same or different; a fourth layer comprising a second anionic nucleic acid, wherein the first anionic and the second anionic nucleic acid can be the same or different; and a fifth layer comprising a third cationic degradable polymer.

In some embodiments, the nanoparticle core comprises an inorganic nanoparticle core. In particular embodiments, the inorganic nanoparticle core comprises a gold nanoparticle core.

In certain embodiments, the first cationic polymer comprises polyethylenimine (PEI). In more certain embodiments, the second cationic polymer comprises a disulfide-reducible poly(amidoamine). In some embodiments, the disulfide-reducible poly(amidoamine) comprises BSS-S3-E7. In yet other embodiments, the third cationic degradable polymer comprises a hydrolytically degradable polymer. In some embodiments, the hydrolytically degradable polymer comprises a poly(β-aminoester). In certain embodiments, the poly(β-aminoester) comprises B4-S4-E7.

In particular embodiments, the first anionic and the second anionic nucleic acid are selected from the group consisting of DNA and siRNA.

### III. Thermo-Sensitive Gels with Heatable Nanoparticles for Dual Hyperthermia and Drug Delivery Systems

Gold nanoparticles have tremendous potential for hyperthermia therapy due to their unique ability to efficiently convert absorbed light into localized heat. The development of near infrared region (NIR) absorbing gold nanoparticles is desirable as NIR light provides penetration through tissue with minimal absorption by hemoglobin and water, allowing for selective laser photothermal therapy of cancer.

Superparamagnetic iron oxide nanoparticles can similarly be remotely heated using an alternating magnetic field. These magnetic particles have an advantage over optical particles in that magnetic fields can penetrate deeper in vivo than NIR light and can be imaged with magnetic resonance.

Poly(ester amine)s are promising drug delivery vehicles due to their degradability and can serve as reservoirs for extracellular delivery of encapsulated drugs, sensors, or inorganic particles. In some embodiment, diacrylate-terminated poly(ester amine)s were crosslinked using acrylate-functionalized monomers and oligomers and a photoinitiator to form polymer gels. At a glass transition temperature, these gels transitioned from a glassy state to a rubbery state, allowing for diffusion of drugs out of the polymer matrix.

MDA-231 are triple negative human breast cancer cells without effective treatment. They are triple negative in that they do not express genes for estrogen, progesterone, or HER2 receptors. Consequently, conventional breast cancer ligand-targeting is ineffective. Thus, a local, gel-based drug release approach to treat breast cancer could be beneficial. The presently disclosed subject matter provides a gel using heatable nanoparticles for hyperthermia and drug therapy. In some embodiments, the presently disclosed drug reservoir system can be placed at the patient's tumor site. The nanoparticles can then be heated magnetically or optically to trigger drug release, destroying tumor cells.

The polymer gels were synthesized to contain a homogeneous distribution of nanoparticles. Using the optical hyperthermia set-up, the ability to control temperature change up to Δ40° C. was demonstrated. Also demonstrated was the ability to control heating using the magnetic hyperthermia set-up through FeCoO nanoparticle concentration and magnetic field strength. The chemotherapeutic drugs doxorubicin and docetaxel also have been encapsulated into the gel system. After finding a suitable gel, viability assays and animal tests will be the next steps in this research.

### IV. Hydrolytic and Bioreducible Polymeric Formulations

Polymer formulations, nanoparticles, and the like, which are suitable for use with the presently disclosed subject matter are disclosed in International PCT Patent Application Publication No. WO/2010/132879 for “Multicomponent Degradable Cationic Polymers,” to Green et al., which is incorporated herein by reference in its entirety, and U.S. patent application Ser. No. 13/272,042 for PEPTIDE/PARTICLE DELIVERY SYSTEM, filed Oct. 12, 2011, which is commonly owned and incorporated herein by reference in its entirety.

In some embodiments, the presently disclosed subject matter generally provides multicomponent degradable cationic polymers. In some embodiments, the presently disclosed polymers have the property of biphasic degradation. Modifications to the polymer structure can result in a change in the release of therapeutic agents, which can occur over multiple time scales. In some embodiments, the presently disclosed polymers include a minority structure, e.g., an endcapping group, which differs from the majority structure comprising most of the polymer backbone. In other embodiments, the bioreducible oligomers form block copolymers with hydrolytically degradable oligomers. In yet other embodiments, the end group/minority structure comprises an amino acid or chain of amino acids, while the backbone degrades hydrolytically and/or is bioreducible.

As described in more detail herein below, small changes in the monomer ratio used during polymerization, in combination with modifications to the chemical structure of the end-capping groups used post-polymerization, can affect the efficacy of delivery of a therapeutic agent to a target. Further, changes in the chemical structure of the polymer, either in the backbone of the polymer or end-capping groups, or both, can change the efficacy of target delivery to a cell. In some embodiments, small changes to the molecular weight of the polymer or changes to the endcapping groups of the polymer, while leaving the main chain, i.e., backbone, of the polymer the same, can enhance or decrease the overall delivery of the target to a cell. Further, the “R” groups that comprise the backbone or main chain of the polymer can be selected to degrade via different biodegradation mechanisms within the same polymer molecule. Such mechanisms include, but are not limited to, hydrolytic, bioreducible, enzymatic, and/or other modes of degradation.

In some embodiments, the presently disclosed compositions can be prepared according to Scheme 2:

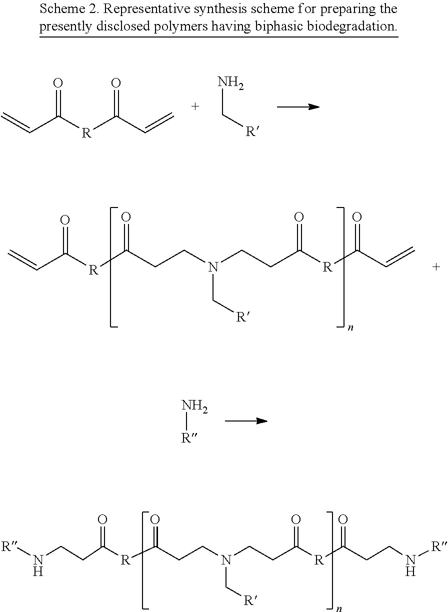

In some embodiments, at least one of the following groups R, R′, and R″ contain reducible linkages and, for many of the presently disclosed materials, additional modes of degradation also are present. More generally, R′ can be any group that facilitates solubility in water and/or hydrogen bonding, for example, OH, NH₂ and SH. Representative degradable linkages include, but are not limited to:

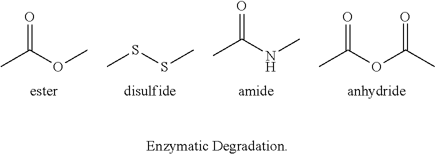

The end group structures, i.e., R″ groups in Scheme 2, for the presently disclosed cationic polymers are distinct and separate from the backbone structures (R) structures, the side chain structures (R′), and end group structures of the intermediate precursor molecule for a given polymeric material.

More particularly, in some embodiments, the presently disclosed subject matter includes a nanoparticle, microparticle, or gel comprising a compound of formula (I):

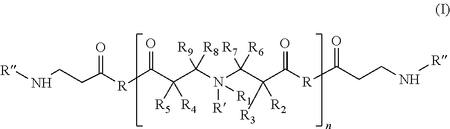

wherein:

n is an integer from 1 to 10,000:

R₁, R₂, R₃, R₄, R₅, R₆, R₇, R₈, and R₉ are each independently selected from the group consisting of hydrogen, branched and unbranched alkyl, branched and unbranched alkenyl, branched and unbranched alkynyl, aryl, halogen, hydroxyl, alkoxy, carbamoyl, carboxyl ester, carbonyldioxyl, amide, thiohydroxyl, alkylthioether, amino, alkylamino, dialkylamino, trialkylamino, cyano, ureido, a substituted alkanoyl group, cyclic, cyclic aromatic, heterocyclic, and aromatic heterocyclic groups, each of which may be substituted with at least one substituent selected from the group consisting of branched or unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiohydroxyl groups;

wherein R₁ can be present or absent and when present the compound of formula (I) further comprises a counter ion selected from the group consisting of chloride, fluoride, bromide, iodide, sulfate, nitrate, fumarate, acetate, carbonate, stearate, laurate, and oleate; and

at least one of R, R′, and R″ comprise a reducible or degradable linkage, and wherein each R, R′, or R″ can independently be the same or different;

under the proviso that when at least one R group comprises an ester linkage of the formula —C(═O)—O— and the compound of formula (I) comprises a poly(beta-amino ester), then the compound of formula (I) must also comprise one or more of the following characteristics:

(a) each R group is different;

(b) each R″ group is different;

(c) each R″ group is not the same as any of R′, R₁, R₂, R₃, R₄, R₅, R₆, R₇, R₈, and R₉;

(d) the R″ groups degrade through a different mechanism than the ester-containing R groups, wherein the degradation of the R″ group is selected from the group consisting of a bioreducible mechanism or an enzymatically degradable mechanism; and/or

(e) the compound of formula (I) comprises a substructure of a larger cross-linked polymer, wherein the larger cross-linked polymer comprises different properties from compound of formula (I);

and one or more peptides selected from the group consisting of an anti-angiogenic peptide, an anti-lymphangiogenic peptide, an anti-tumorigenic peptide, and an anti-permeability peptide.

In some embodiments of the nanoparticle, microparticle, or gel n is an integer from 1 to 1,000; in some embodiments, n is an integer from 1 to 100; in some embodiments, n is an integer from 1 to 30; in some embodiments, n is an integer from 5 to 20; in some embodiments, n is an integer from 10 to 15; and in some embodiments, n is an integer from 1 to 10.

In particular embodiments, the reducible or degradable linkage comprising R, R′, and R″ is selected from the group consisting of an ester, a disulfide, an amide, an anhydride or a linkage susceptible to enzymatic degradation, subject to the proviso provided hereinabove.

In more particular embodiments, R comprises a backbone of a diacrylate selected from the group consisting of:

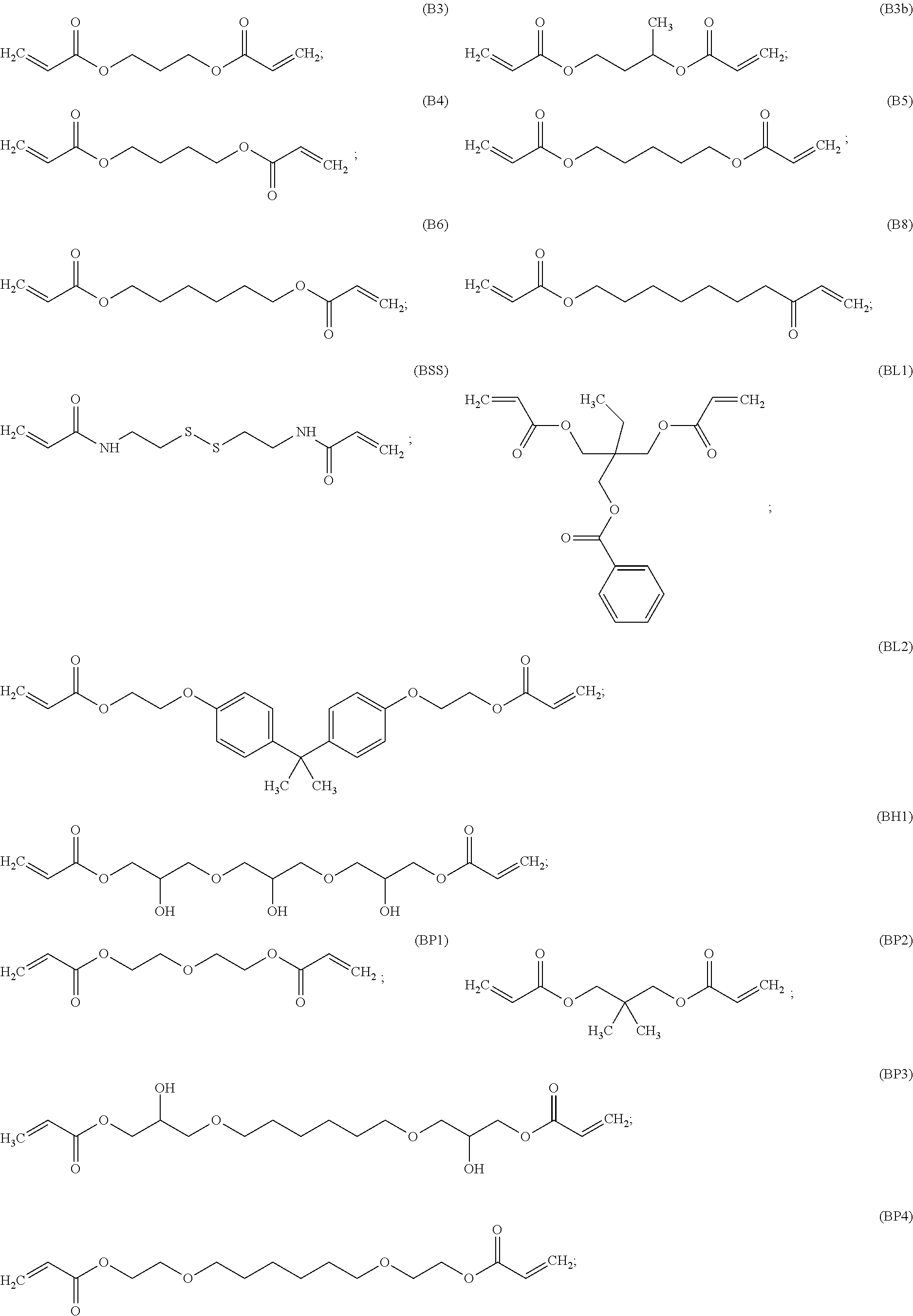

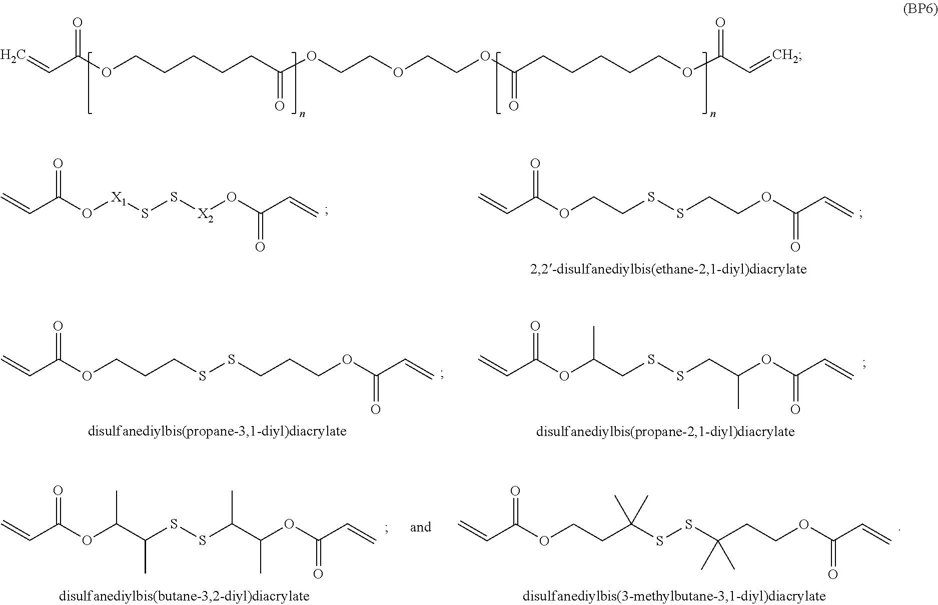

In some embodiments, wherein R′ comprises a side chain derived from compound selected from the group consisting of:

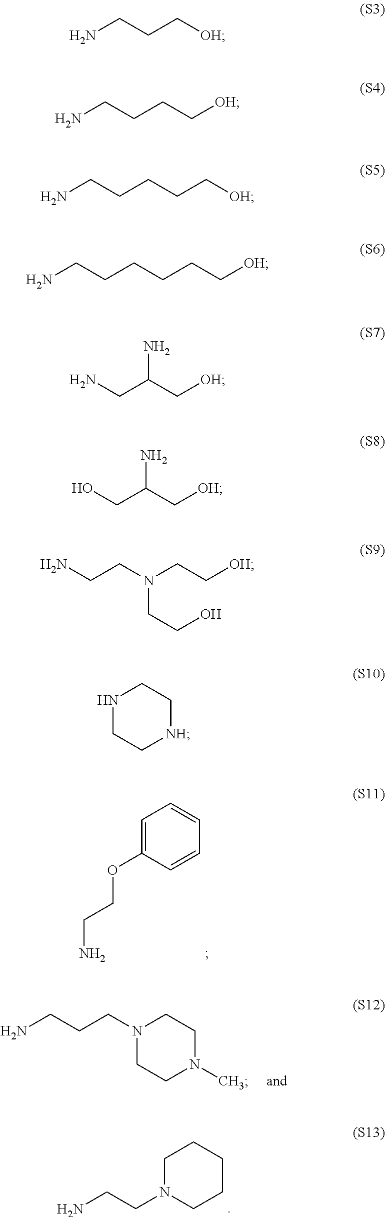

In some embodiments, R″ comprises an end group derived from a compound selected from the group consisting of

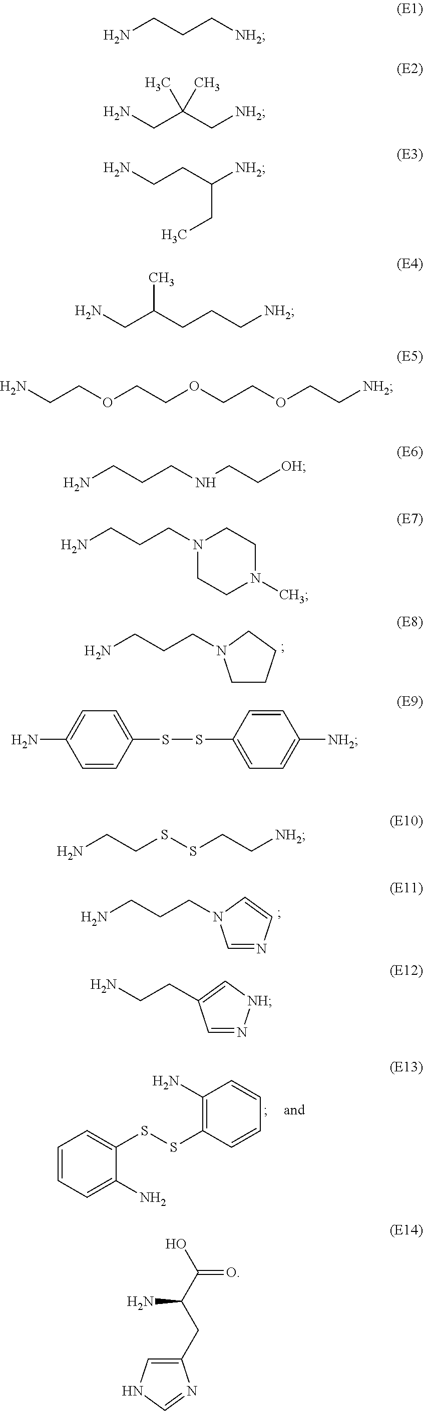

In other embodiments, the compound of formula (I) is subject to the further proviso that if at least one R group comprises an ester linkage, then the R″ groups impart one or more of the following characteristics to the compound of formula (I): independent control of cell-specific uptake and/or intracellular delivery of a particle; independent control of endosomal buffering and endosomal escape; independent control of DNA release: triggered release of an active agent; modification of a particle surface charge; increased diffusion through a cytoplasm of a cell; increased active transport through a cytoplasm of a cell; increased nuclear import within a cell; increased transcription of an associated DNA within a cell; increased translation of an associated DNA within a cell; increased persistence of an associated therapeutic agent within a cell, wherein the therapeutic agent is selected from the group consisting of DNA, RNA, a peptide or a protein.

More particularly, any poly(beta-amino ester) specifically disclosed or claimed in U.S. Pat. No. 6,998,115; U.S. Pat. No. 7,427,394; U.S. patent application publication no. US2005/0265961; and U.S. patent publication no. US2010/0036084, each of which is incorporated herein by reference in its entirety, is explicitly excluded from the presently disclosed compounds of formula (I). In particular, the poly(beta-amino ester)s disclosed in U.S. Pat. No. 6,998,115; U.S. Pat. No. 7,427,394; U.S. patent application publication no. US2005/0265961; and U.S. patent publication no. US2010/0036084 are symmetrical, i.e., both R groups as defined in formula (I) herein are the same. In certain embodiments of the presently disclosed compounds of formula (I), when at least one R comprises an ester linkage, the two R groups of formula (I) are not the same, i.e., in such embodiments, the compounds of formula (I) are not symmetrical.

In particular embodiments, the reducible or degradable linkage comprising R, R′, and R″ is selected from the group consisting of an ester, a disulfide, an amide, an anhydride or a linkage susceptible to enzymatic degradation, subject to the above-mentioned provisos.

Further, in some embodiments of the compound of formula (I), n is an integer from 1 to 1,000; in other embodiments, n is an integer from 1 to 100; in other embodiments, n is an integer from 1 to 30; in other embodiments, n is an integer from 5 to 20; in other embodiments, n is an integer from 10 to 15: and in other embodiments, n is an integer from 1 to 10.

In some embodiments, R″ can be an oligomer as described herein, e.g., one fully synthesized primary amine-terminated oligomer, and can be used as a reagent during the second reaction step of Scheme 2. This process can be repeated iteratively to synthesize increasingly complex molecules.

In other embodiments, R″ can comprise a larger biomolecule including, but not limited to, poly(ethyleneglycol) (PEG), a targeting ligand, including, but not limited to, a sugar, a small molecule, an antibody, an antibody fragment, a peptide sequence, or other targeting moiety known to one skilled in the art; a labeling molecule including, but not limited to, a small molecule, a quantum dot, a nanoparticle, a fluorescent molecule, a luminescent molecule, a contrast agent, and the like; and a branched or unbranched, substituted or unsubstituted alkyl chain.

In some embodiments, the branched or unbranched, substituted or unsubstituted alkyl chain is about 2 to about 5 carbons long; in some embodiments, the alkyl chain is about 6 to about 8 carbons long; in some embodiments, the alkyl chain is about 9 to about 12 carbons long; in some embodiments, the alkyl chain is about 13 to about 18 carbons long; in some embodiments, the alkyl chain is about 19 to about 30 carbons long; in some embodiments, the alkyl chain is greater than about carbons long.

In certain embodiments, both R″ groups, i.e., the end groups of the polymer, comprise alkyl chains. In other embodiments, only one R″ group comprises an alkyl chain. In some embodiments, at least one alkyl chain is terminated with an amino (NH₂) group. In other embodiments, the at least one alkyl chain is terminated with a hydroxyl (OH) group.

In some embodiments, the PEG has a molecular weight of about 5 kDa or less; in some embodiments, the PEG has a molecular weight of about 5 kDa to about 10 kDa; in some embodiments, the PEG has a molecular weight of about 10 kDa to about 20 kDa; in some embodiments, the PEG has a molecular weight of about 20 kDa to about 30 kDa; in some embodiments, the PEG is greater than 30 kDa. In certain embodiments, both R″ groups comprise PEG. In other embodiments, only one R″ group comprises PEG.

Further, in some embodiments, one R″ group is PEG and the other R″ group is a targeting ligand and/or labeling molecule as defined herein above. In other embodiments, one R″ group is an alkyl chain and the other R″ group is a targeting ligand and/or labeling molecule.

Representative monomers used to synthesize the presently disclosed cationic polymers include, but are not limited to, those provided immediately herein below. The presently disclosed subject matter is not limited to the representative monomers disclosed herein, but also includes other structures that one skilled in the art could use to create similar biphasic degrading cationic polymers. For each type of cargo, a particular biodegradable polymer can be tuned through varying the constituent monomers used to form the backbone (designated as “B” groups), side-chains (designated as “S” groups), and end-groups (designated as “E” groups) of the polymer.

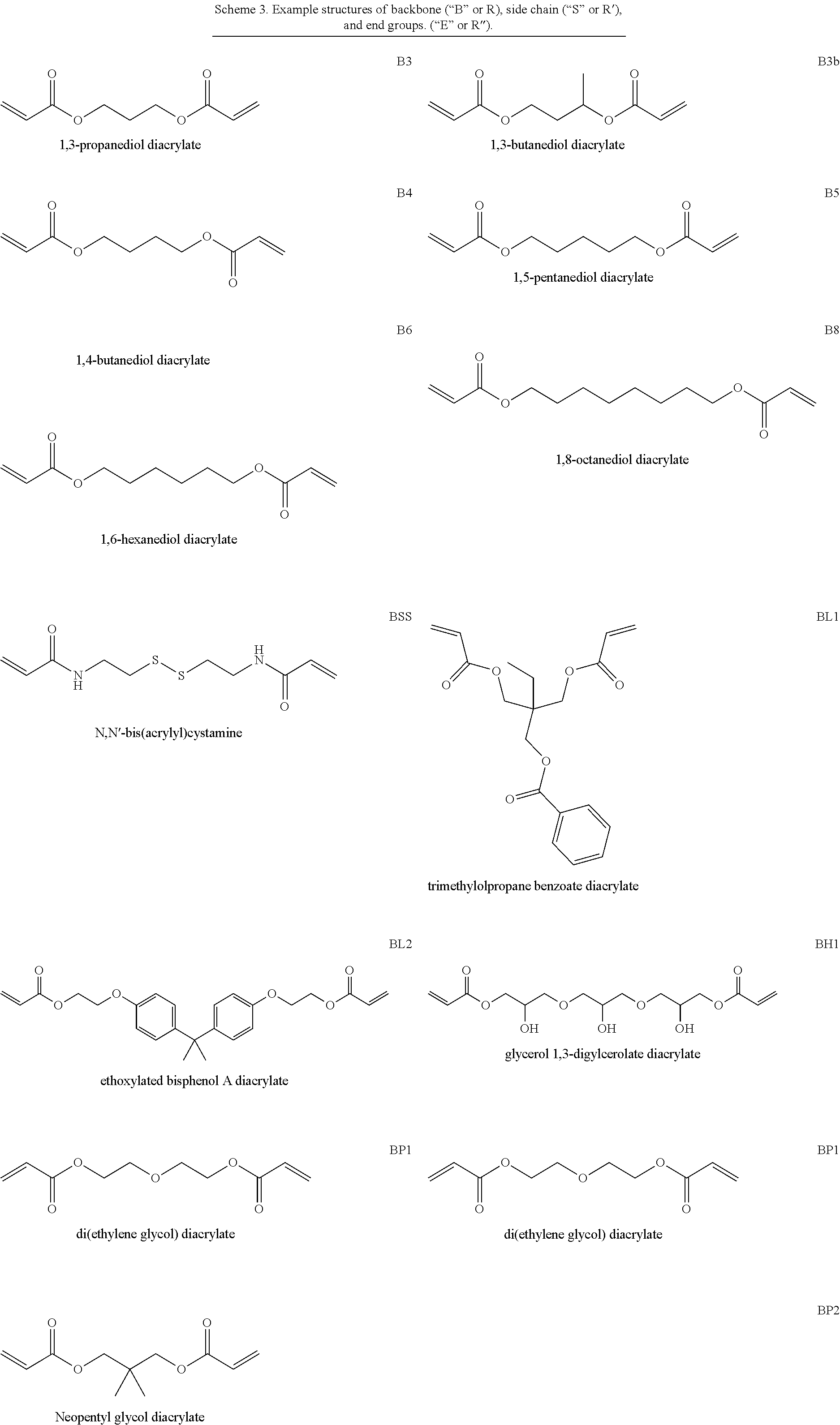

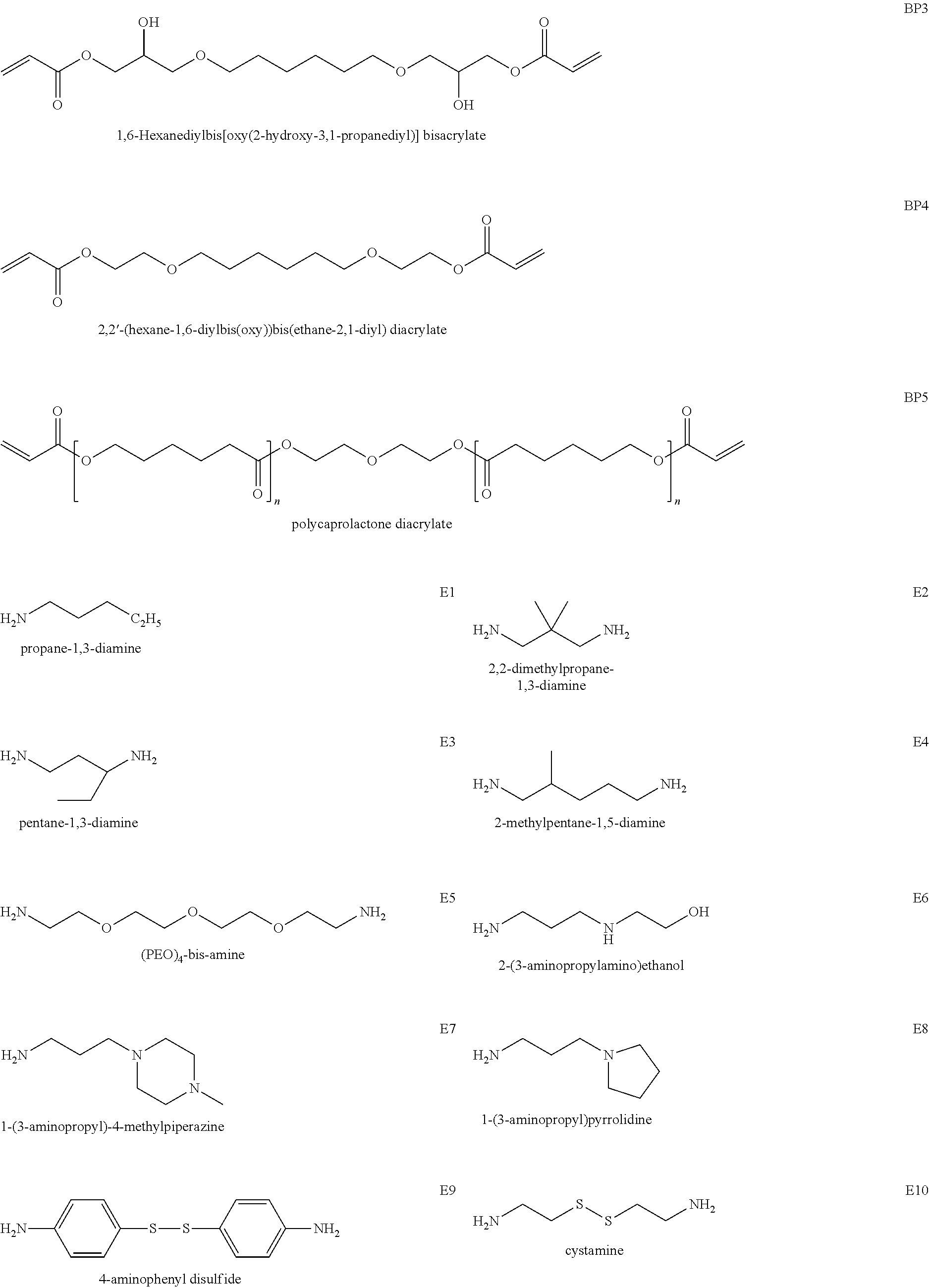

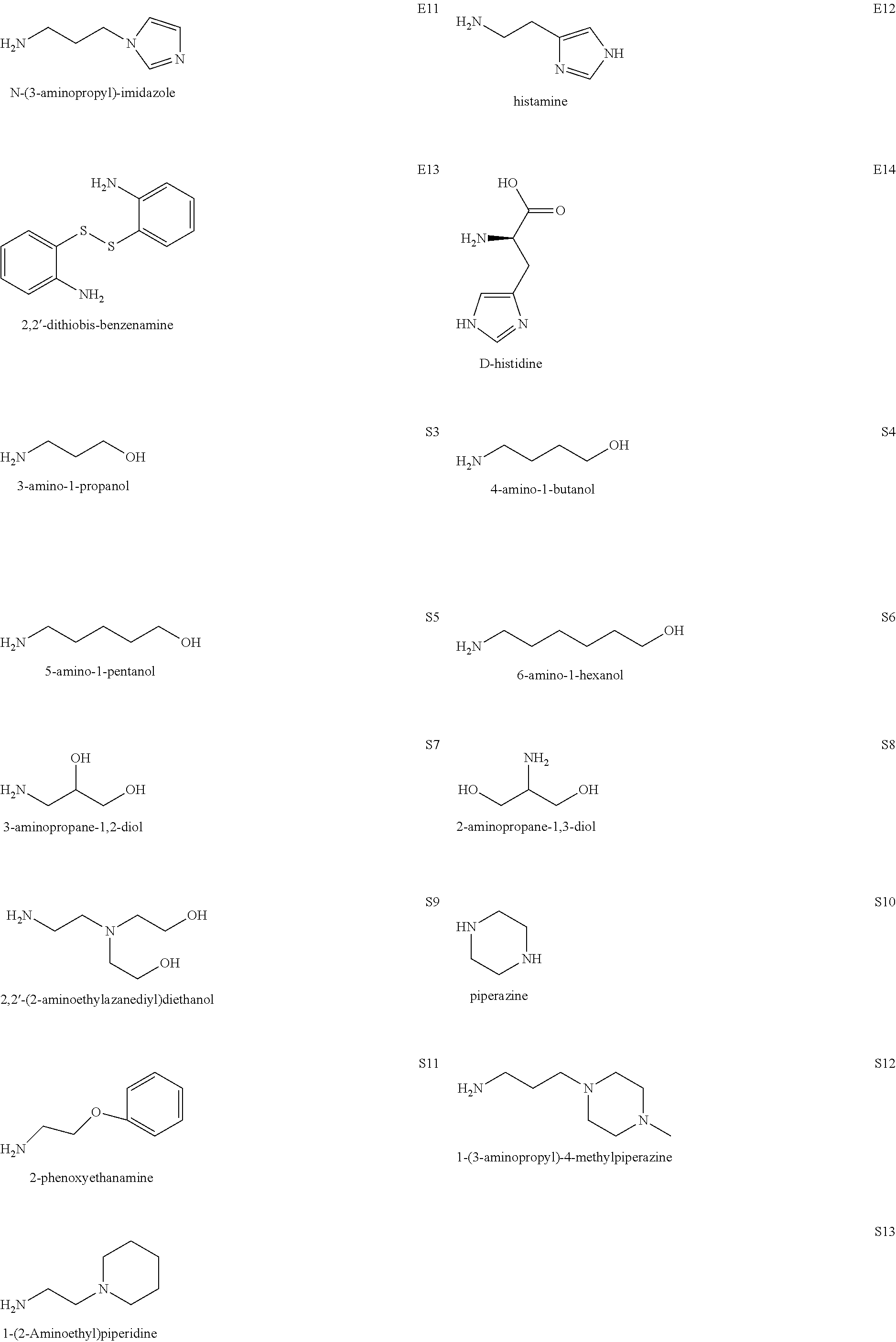

In particular embodiments, as depicted in Scheme 4, the presently disclosed cationic polymers comprise a polyalcohol structure, i.e., the side chain represented by R′ in Scheme 2 comprises an alcohol.

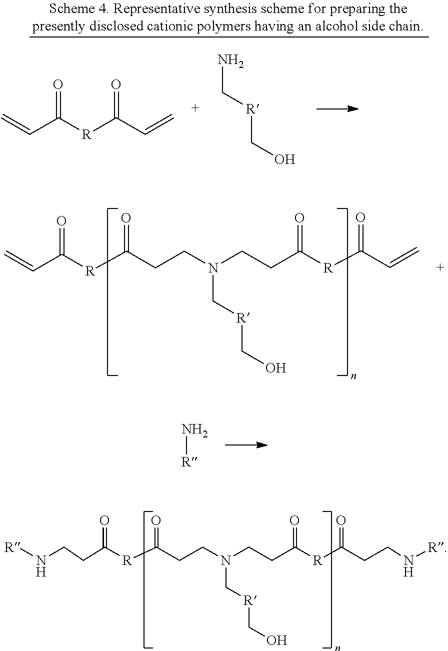

In such embodiments, the end group structures (R″) and the backbone structures (R) are defined as above and the side chain must contain at least one hydroxyl (OH) group.

In yet other embodiments, the presently disclosed cationic polymer comprises a specific poly(ester amine) structure with secondary non-hydrolytic modes of degradation. In such embodiments, the cationic polymer comprises a polyester that degrades through ester linkages (hydrolytic degradation) that is further modified to comprise bioreducible groups as end (R″) groups.

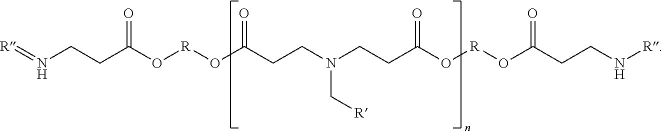

Representative bioreducible end groups in such embodiments include, but are not limited to:

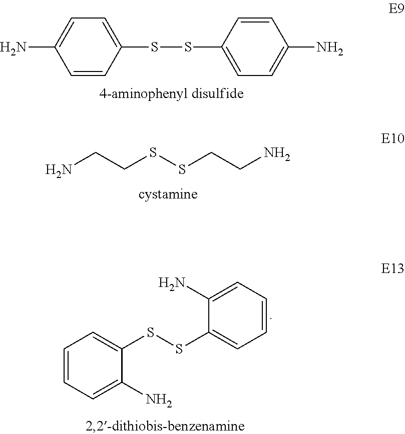 [SMILES: Nc1ccc(SSc2ccc(N)cc2)cc1 ; NCCSSCCN ; Nc1ccccc1SSc1ccccc1N]

In some embodiments, the presently disclosed cationic polymer comprises a specific poly(ester amine alcohol) structure with secondary non-hydrolytic modes of degradation. In such embodiments, the cationic polymer comprises a specific structure where a polyester that degrades through ester linkages (hydrolytic degradation) is modified to contain bioreducible groups as end groups.

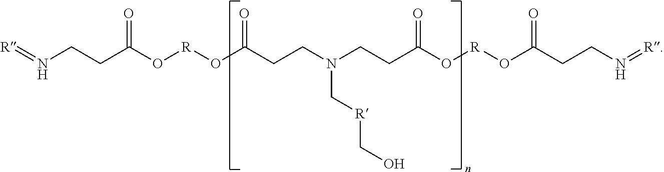

In yet other embodiments, the presently disclosed cationic polymer comprises a specific poly(amido amine) structure having disulfide linking groups in the polymer backbone and an independent, non-reducible amine contacting group at the terminal ends of the polymer.

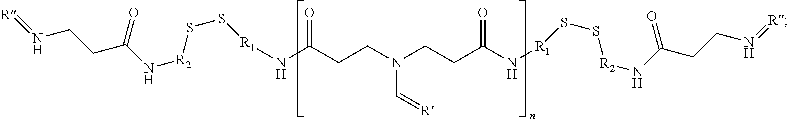

In such embodiments, R₁ and R₂ are alkyl chains. In some embodiments, the alkyl chain is 1-2 carbons long; in some embodiments, the alkyl chain is 3-5 carbons long; in some embodiments, the alkyl chain is 6-8 carbons long; in some embodiments, the alkyl chain is 9-12 carbons long; in some embodiments, the alkyl chain is 13-18 carbons long; in some embodiments, the alkyl chain is 19-30 carbons long; and in some embodiments, the alkyl chain is greater than 30 carbons long

Suitable non-reducible amino R″ groups for such embodiments include, but are not limited to:

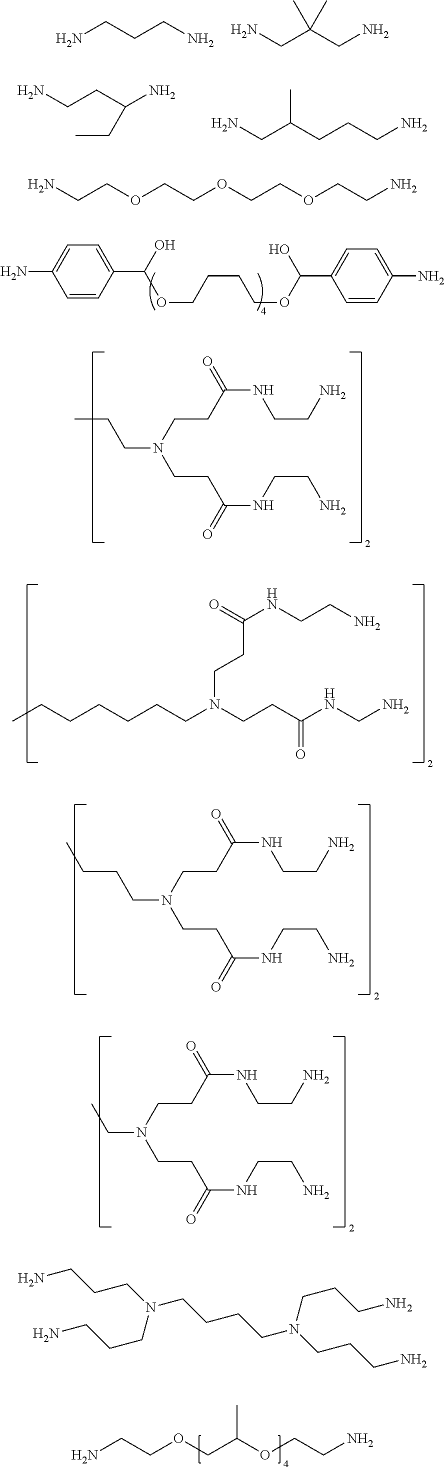

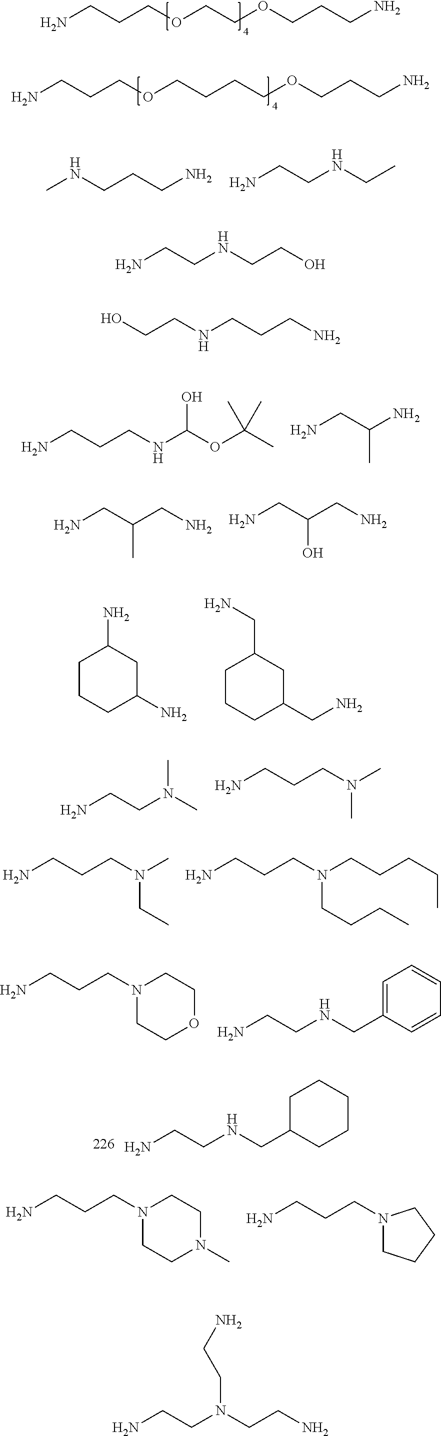

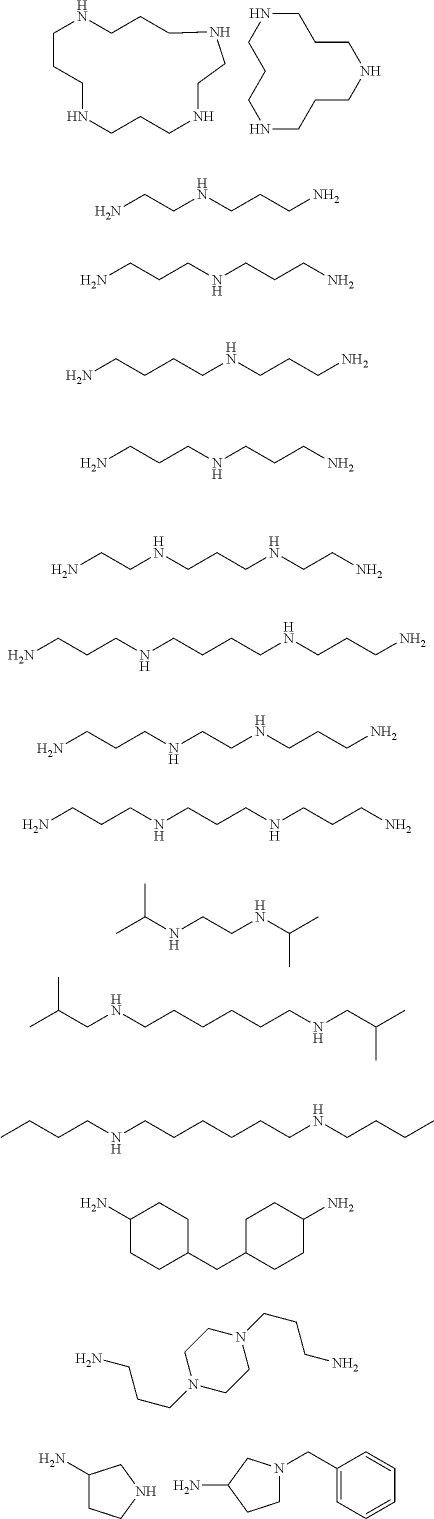

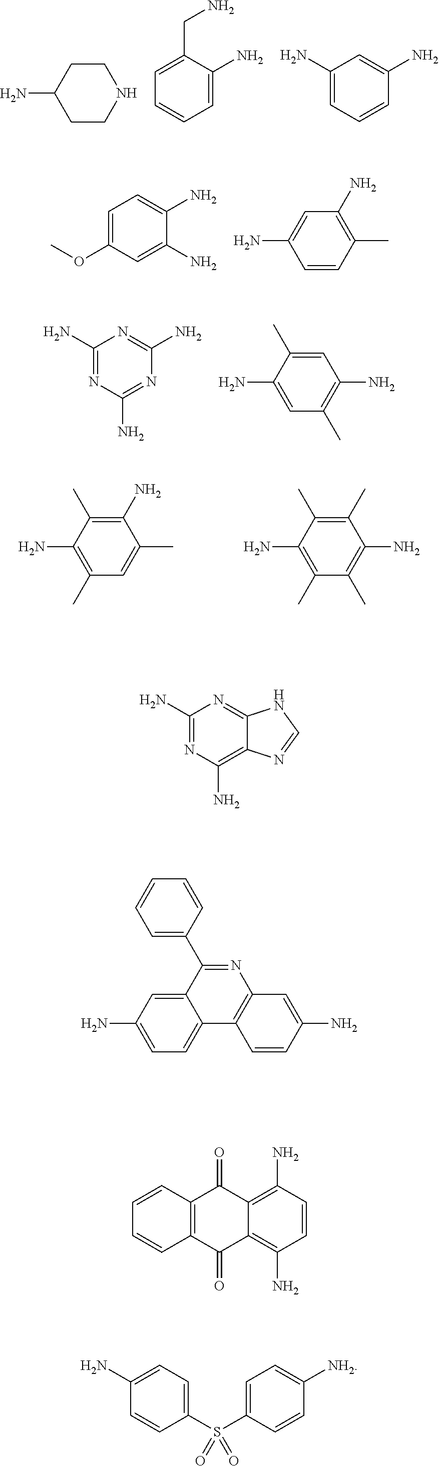

In other embodiments, the presently disclosed cationic polymers comprise a specific poly(amido amine alcohol) structure having disulfide linking groups in the polymer backbone and an independent non-reducible amine contacting group at the terminal ends of the polymer.

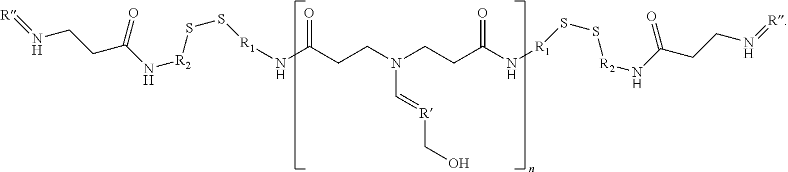

In yet other embodiments, the presently disclosed cationic polymer comprises a copolymer of representative oligomers as described hereinabove. Such embodiments include, but are not limited to, a poly(amido amine) structure having disulfides in the polymer backbone and an independently degradable (non-reducible) group at least one end of the polymer. Such embodiments also include using a cross-linker to add bioreducible linkages to hydrolytically degradable materials and also provide for higher molecular weight materials. A representative example of this embodiment, along with suitable monomers is as follows:

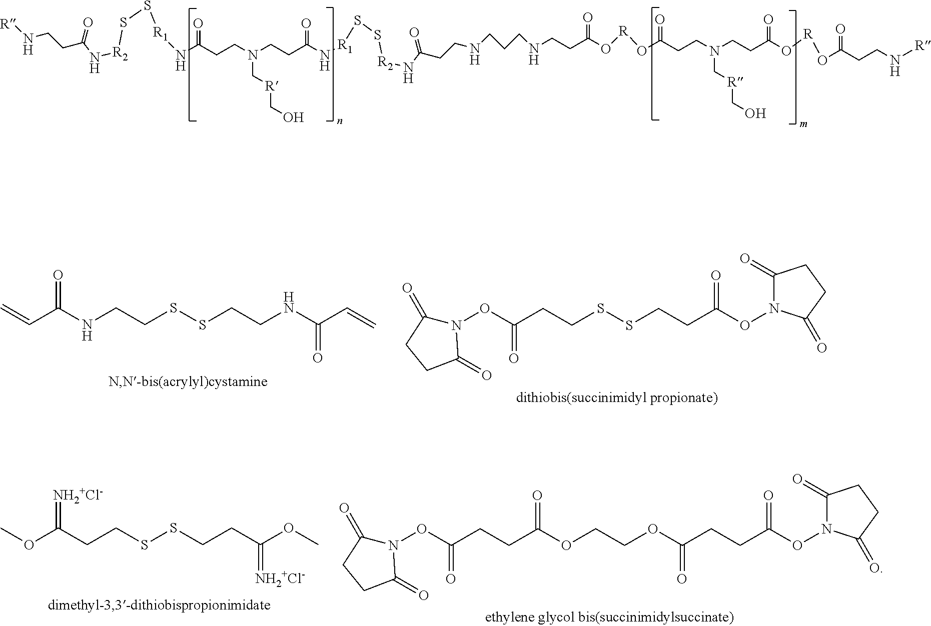

In particular embodiments, the presently disclosed polymer is selected from the group consisting of:

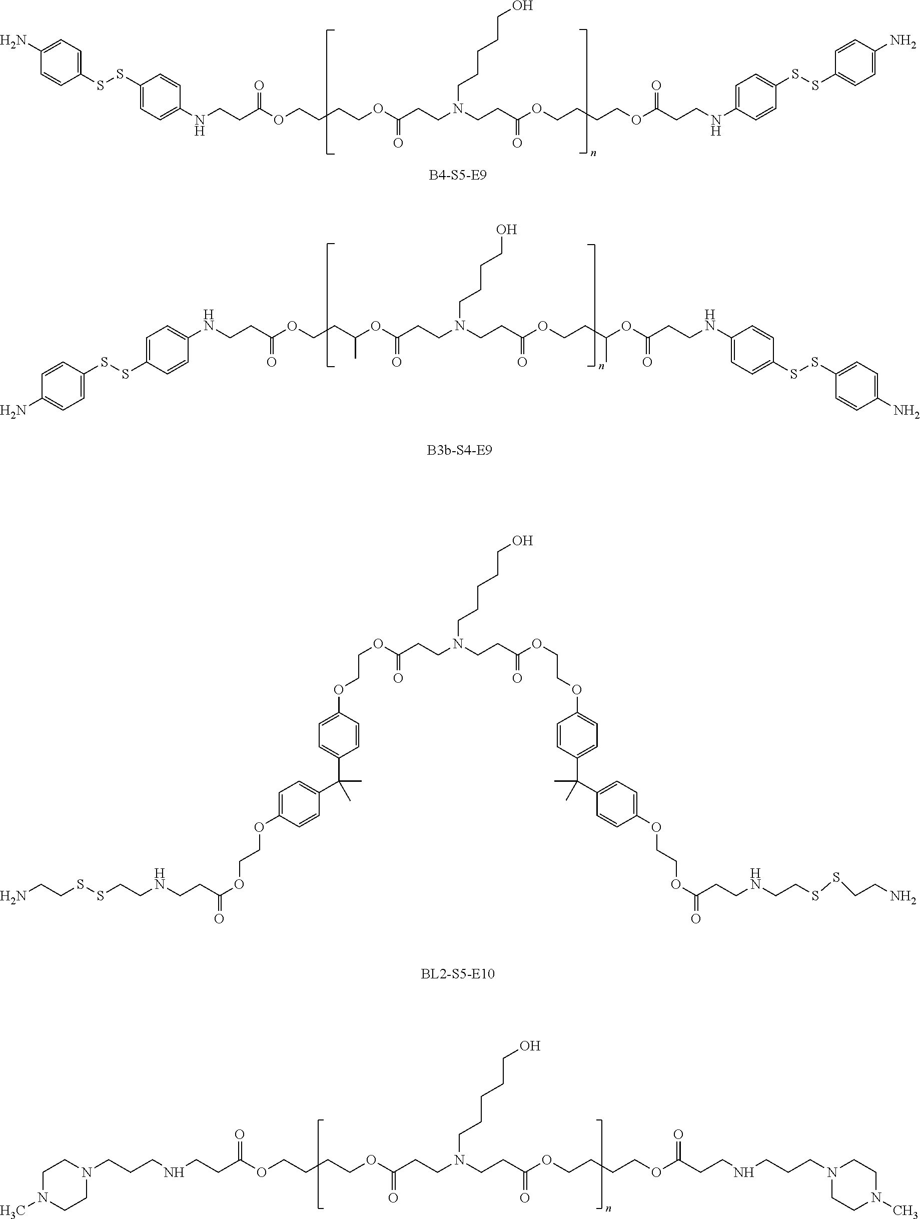

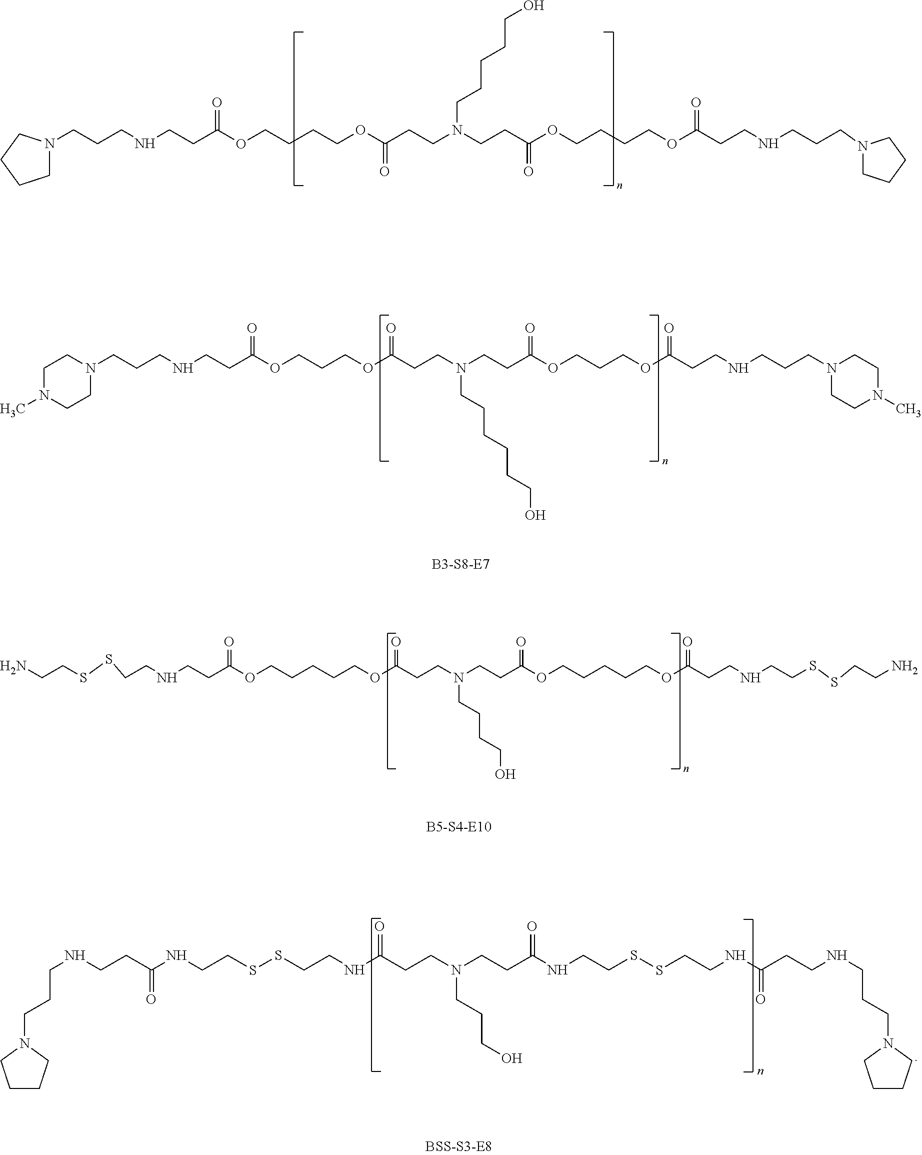

Further aspects of the presently disclosed subject matter include: (a) the R substituent groups that make up the presently disclosed polymers degrade via different biodegradation mechanisms within the same polymer. These biodegradation mechanisms can include hydrolytic, bioreducible, enzymatic, and/or other modes of degradation; (b) the ends of the polymer include a minority structure that differs from the majority structure that comprises most of the polymer backbone; (c) in several embodiments, the side chain molecules contain hydroxyl (OH)/alcohol groups.

In some embodiments: (a) the backbone is bioreducible and the end groups of the polymer degrade hydrolytically; (b) the backbone degrades hydrolytically and the end groups are bioreducible; and (c) hydrolytically degradable oligomers are cross-linked with a bioreducible cross-linker; (d) bioreducible oligomers form block copolymers with hydrolytically degradable oligomers; and (e) the end group/minority structure comprises an amino acid or chain of amino acids, whereas the backbone degrades hydrolytically and/or is bioreducible.

One way to synthesize the presently disclosed materials is by the conjugate addition of amine-containing molecules to acrylates or acrylamides. This reaction can be done neat or in a solvent, such as DMSO or THF. Reactions can take place at a temperature ranging from about room temperature up to about 90° C. and can have a duration from about a few hours to about a few weeks. The presently disclosed methods can be used to create linear or branched polymers. In some embodiments, the molecular weight (MW) has a range from about 1 kDa to about 5 kDa, in other embodiments, the MW has a range from about 5 kDa to about 10 kDa, in other embodiments the MW has a range from about 10 kDa to about 15 kDa, in other embodiments, the MW has a range from about 15 kDa to about 25 kDa, in other embodiments, the MW has a range from about 25 kDa to about 50 kDa, and in other embodiments, the MW has a range from about 50 kDa to about 100 kDa. In other embodiments, the polymer forms a network, gel, and/or scaffold of apparent molecular weight greater than 100 kDa.

### V. DEFINITIONS

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to the presently disclosed compounds are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted, for example, with fluorine at one or more positions).

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

A named “R” or group will generally have the structure that is recognized in the art as corresponding to a group having that name, unless specified otherwise herein. For the purposes of illustration, certain representative “R” groups as set forth above are defined below.

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, iso-propyl, cyclopropyl, allyl, vinyl, n-butyl, tert-butyl, ethynyl, cyclohexyl, methoxy, diethylamino, and the like.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom. Representative alkyl groups include, but are not limited to, methyl, ethyl, n-propyl, isopropyl, n-butyl, isobutyl, sec-butyl, tert-butyl, n-pentyl, sec-pentyl, iso-pentyl, neopentyl, n-hexyl, sec-hexyl, n-heptyl, n-octyl, n-decyl, n-undecyl, dodecyl, and the like, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁.s alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of N, O, and S, and optionally can include one or more double bonds. The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The term “alkenyl” as used herein refers to a monovalent group derived from a C₁₋₂₀ inclusive straight or branched hydrocarbon moiety having at least one carbon-carbon double bond by the removal of a single hydrogen atom. Alkenyl groups include, for example, ethenyl (i.e., vinyl), propenyl, butenyl, 1-methyl-2-buten-1-yl, and the like.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₁₋₂₀ hydrocarbon of a designed number of carbon atoms containing at least one carbon-carbon triple bond. Examples of “alkynyl” include ethynyl, 2-propynyl (propargyl), 1-propyne, 3-hexyne, and the like.

“Alkylene” refers to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, alkenyl, alkynyl, aryl, substituted aryl, aralkyl, hydroxyl, alkoxyl, aryloxyl, aralkyloxyl, carboxyl, acyl, halo, haloalkyl, nitro, alkoxycarbonyl, aryloxycarbonyl, aralkoxycarbonyl, acyloxyl, amino, alkylamino, dialkylamino, trialkylamino, acylamino, aroylamino, carbamoyl, cyano, alkylcarbamoyl, dialkylcarbamoyl, carboxyaldehyde, carboxyl, alkoxycarbonyl, carboxamide, arylthio, alkylthio, alkylene, thioalkoxyl, and mercapto.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

The terms “heteroaryl” and “aromatic heterocycle” and “aromatic heterocyclic” are used interchangeably herein and refer to a cyclic aromatic radical having from five to ten ring atoms of which one ring atom is selected from sulfur, oxygen, and nitrogen; zero, one, or two ring atoms are additional heteroatoms independently selected from sulfur, oxygen, and nitrogen; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like. Aromatic heterocyclic groups can be unsubstituted or substituted with substituents selected from the group consisting of branched and unbranched alkyl, alkenyl, alkynyl, haloalkyl, alkoxy, thioalkoxy, amino, alkylamino, dialkylamino, trialkylamino, acylamino, cyano, hydroxy, halo, mercapto, nitro, carboxyaldehyde, carboxy, alkoxycarbonyl, and carboxamide.

Specific heterocyclic and aromatic heterocyclic groups that may be included in the presently disclosed compounds include: 3-methyl-4-(3-methylphenyl)piperazine, 3 methylpiperidine, 4-(bis-(4-fluorophenyl)methyl)piperazine, 4-(diphenylmethyl)piperazine, 4(ethoxycarbonyl)piperazine, 4-(ethoxycarbonylmethyl)piperazine, 4-(phenylmethyl)piperazine, 4-(1-phenylethyl)piperazine, 4-(1,1-dimethylethoxycarbonyl)piperazine, 4-(2-(bis-(2-propenyl) amino)ethyl)piperazine, 4-(2-(diethylamino)ethyl)piperazine, 4-(2-chlorophenyl)piperazine, 4(2-cyanophenyl)piperazine, 4-(2-ethoxyphenyl)piperazine, 4-(2-ethylphenyl)piperazine, 4-(2-fluorophenyl)piperazine, 4-(2-hydroxyethyl)piperazine, 4-(2-methoxyethyl)piperazine, 4-(2-methoxyphenyl)piperazine, 4-(2-methylphenyl)piperazine, 4-(2-methylthiophenyl) piperazine, 4(2-nitrophenyl)piperazine, 4-(2-nitrophenyl)piperazine, 4-(2-phenylethyl)piperazine, 4-(2-pyridyl)piperazine, 4-(2-pyrimidinyl)piperazine, 4-(2,3-dimethylphenyl)piperazine, 4-(2,4-difluorophenyl) piperazine, 4-(2,4-dimethoxyphenyl)piperazine, 4-(2,4-dimethylphenyl)piperazine, 4-(2,5-dimethylphenyl)piperazine, 4-(2,6-dimethylphenyl)piperazine, 4-(3-chlorophenyl)piperazine, 4-(3-methylphenyl)piperazine, 4-(3-trifluoromethylphenyl)piperazine, 4-(3,4-dichlorophenyl)piperazine, 4-3,4-dimethoxyphenyl)piperazine, 4-(3,4-dimethylphenyl)piperazine, 4-(3,4-methylenedioxyphenyl)piperazine, 4-(3,4,5-trimethoxyphenyl)piperazine, 4-(3,5-dichlorophenyl)piperazine, 4-(3,5-dimethoxyphenyl)piperazine, 4-(4-(phenylmethoxy)phenyl)piperazine, 4-(4-(3,1-dimethylethyl)phenylmethyl)piperazine, 4-(4-chloro-3-trifluoromethylphenyl)piperazine, 4-(4-chlorophenyl)-3-methylpiperazine, 4-(4-chlorophenyl)piperazine, 4-(4-chlorophenyl)piperazine, 4-(4-chlorophenylmethyl)piperazine, 4-(4-fluorophenyl)piperazine, 4-(4-methoxyphenyl)piperazine, 4-(4-methylphenyl)piperazine, 4-(4-nitrophenyl)piperazine, 4-(4-trifluoromethylphenyl)piperazine, 4-cyclohexylpiperazine, 4-ethylpiperazine, 4-hydroxy-4-(4-chlorophenyl)methylpiperidine, 4-hydroxy-4-phenylpiperidine, 4-hydroxypyrrolidine, 4-methylpiperazine, 4-phenylpiperazine, 4-piperidinylpiperazine, 4-(2-furanyl)carbonyl)piperazine, 4-((1,3-dioxolan-5-yl)methyl)piperazine, 6-fluoro-1,2,3,4-tetrahydro-2-methylquinoline, 1,4-diazacylcloheptane, 2,3-dihydroindolyl, 3,3-dimethylpiperidine, 4,4-ethylenedioxypiperidine, 1,2,3,4-tetrahydroisoquinoline, 1,2,3,4-tetrahydroquinoline, azacyclooctane, decahydroquinoline, piperazine, piperidine, pyrrolidine, thiomorpholine, and triazole. The heteroaryl ring can be fused or otherwise attached to one or more heteroaryl rings, aromatic or non-aromatic hydrocarbon rings, or heterocycloalkyl rings. A structure represented generally by the formula:

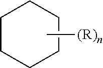

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

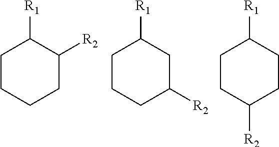

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

When a named atom of an aromatic ring or a heterocyclic aromatic ring is defined as being “absent,” the named atom is replaced by a direct bond.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as an acetylfuran and a phenacyl group. Specific examples of acyl groups include acetyl and benzoyl.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, t-butoxyl, and n-pentoxyl, neopentoxy, n-hexoxy, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl.

“Alkoxycarbonyl” refers to an alkyl-O—CO— group. Exemplary alkoxycarbonyl groups include methoxycarbonyl, ethoxycarbonyl, butyloxycarbonyl, and t-butyloxycarbonyl.

“Aryloxycarbonyl” refers to an aryl-O—CO— group. Exemplary aryloxycarbonyl groups include phenoxy- and naphthoxy-carbonyl.

“Aralkoxycarbonyl” refers to an aralkyl-O—CO— group. An exemplary aralkoxycarbonyl group is benzyloxycarbonyl.

“Carbamoyl” refers to an amide group of the formula —CONH₂. “Alkylcarbamoyl” refers to a R′RN—CO— group wherein one of R and R′ is hydrogen and the other of R and R′ is alkyl and/or substituted alkyl as previously described. “Diallkylcarbamoyl” refers to a R′RN—CO— group wherein each of R and R′ is independently alkyl and/or substituted alkyl as previously described.

The term carbonyldioxyl, as used herein, refers to a carbonate group of the formula —O—CO-—OR.

“Acyloxyl” refers to an acyl-O— group wherein acyl is as previously described.

The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

The terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, iso-propylamino, piperidino, trimethylamino, and propylamino.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH—group wherein acyl is as previously described. “Aroylamino” refers to an aroyl-NH—group wherein aroyl is as previously described.

The term “carbonyl” refers to the —(C═O)— group.

The term “carboxyl” refers to the —COOH group. Such groups also are referred to herein as a “carboxylic acid” moiety.

The terms “halo,” “halide,” or “halogen” as used herein refer to fluoro, chloro, bromo, and iodo groups.

The term “hydroxyl” refers to the —OH group.

The term “hydroxyalkyl” refers to an alkyl group substituted with an —OH group.

The term “mercapto” refers to the —SH group.

The term “oxo” refers to a compound described previously herein wherein a carbon atom is replaced by an oxygen atom.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

The term ureido refers to a urea group of the formula —NH—CO—NH₂.

Throughout the specification and claims, a given chemical formula or name shall encompass all tautomers, congeners, and optical- and stereoisomers, as well as racemic mixtures where such isomers and mixtures exist.

As used herein the term “monomer” refers to a molecule that can undergo polymerization, thereby contributing constitutional units to the essential structure of a macromolecule or polymer.

A “polymer” is a molecule of high relative molecule mass, the structure of which essentially comprises the multiple repetition of unit derived from molecules of low relative molecular mass, i.e., a monomer.

As used herein, an “oligomer” includes a few monomer units, for example, in contrast to a polymer that potentially can comprise an unlimited number of monomers. Dimers, trimers, and tetramers are non-limiting examples of oligomers.

Further, as used herein, the term “nanoparticle,” refers to a particle having at least one dimension in the range of about 1 nm to about 1000 nm, including any integer value between 1 nm and 1000 nm (including about 1, 2, 5, 10, 20, 50, 60, 70, 80, 90, 100, 200, 500, and 1000 nm and all integers and fractional integers in between). In some embodiments, the nanoparticle has at least one dimension, e.g., a diameter, of about 100 nm. In some embodiments, the nanoparticle has a diameter of about 200 nm. In other embodiments, the nanoparticle has a diameter of about 500 nm. In yet other embodiments, the nanoparticle has a diameter of about 1000 nm (1 μm). In such embodiments, the particle also can be referred to as a “microparticle. Thus, the term “microparticle” includes particles having at least one dimension in the range of about one micrometer (Am), i.e., 1×10⁶ meters, to about 1000 μm. The term “particle” as used herein is meant to include nanoparticles and microparticles.

It will be appreciated by one of ordinary skill in the art that nanoparticles suitable for use with the presently disclosed methods can exist in a variety of shapes, including, but not limited to, spheroids, rods, disks, pyramids, cubes, cylinders, nanohelixes, nanosprings, nanorings, rod-shaped nanoparticles, arrow-shaped nanoparticles, teardrop-shaped nanoparticles, tetrapod-shaped nanoparticles, prism-shaped nanoparticles, and a plurality of other geometric and non-geometric shapes. In particular embodiments, the presently disclosed nanoparticles have a spherical shape.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc.

“Biocompatible”: The term “biocompatible”, as used herein is intended to describe compounds that are not toxic to cells. Compounds are “biocompatible” if their addition to cells in vitro results in less than or equal to 20% cell death, and their administration in vivo does not induce inflammation or other such adverse effects.

“Biodegradable”: As used herein, “biodegradable” compounds are those that, when introduced into cells, are broken down by the cellular machinery or by hydrolysis into components that the cells can either reuse or dispose of without significant toxic effect on the cells (i.e., fewer than about 20% of the cells are killed when the components are added to cells in vitro). The components preferably do not induce inflammation or other adverse effects in vivo. In certain preferred embodiments, the chemical reactions relied upon to break down the biodegradable compounds are uncatalyzed.

“Effective amount”: In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, and the like.

“Peptide” or “protein”: A “peptide” or “protein” comprises a string of at least three amino acids linked together by peptide bonds. The terms “protein” and “peptide” may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polynucleotide” or “oligonucleotide”: Polynucleotide or oligonucleotide refers to a polymer of nucleotides. Typically, a polynucleotide comprises at least three nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, 0(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Small molecule”: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. Known naturally-occurring small molecules include, but are not limited to, penicillin, erythromycin, taxol, cyclosporin, and rapamycin. Known synthetic small molecules include, but are not limited to, ampicillin, methicillin, sulfamethoxazole, and sulfonamides.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1

### Nano-Gold/Degradable Polymer Hybrid Nanoparticles for Co-Delivery of DNA and siRNA

### Methods

### AuNP Synthesis and Characterization

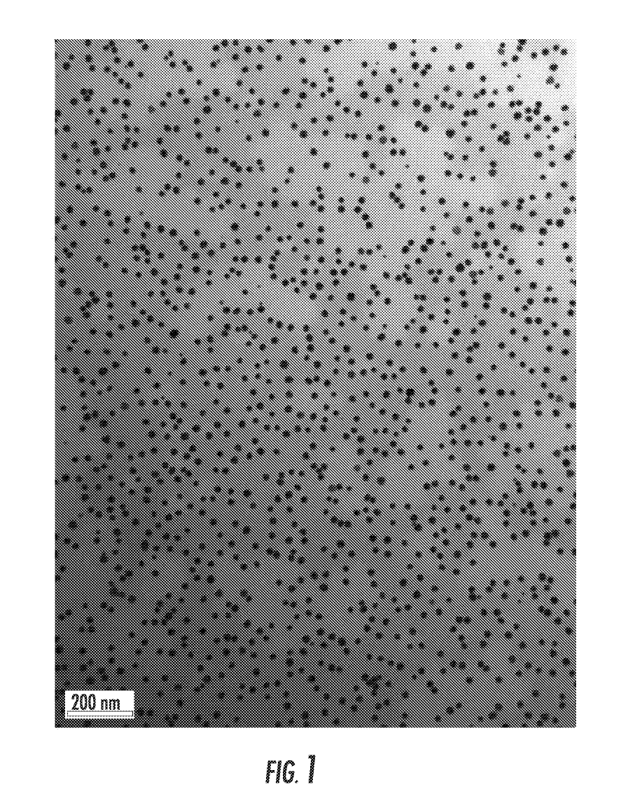

Tetrachloroauric acid was dissolved in ultra pure distilled water forming a 0.01% solution (solution I). A solution of tri-sodium citrate was then made at a 1% concentration (solution II). The ratio of solution I to solution II controls the size of the AuNPs during formation. Solution I is brought to a vigorous boil using a mineral bath and a reflux condenser. Solution II is then added and the two solutions are allowed to boil for 6 min. A I:II solution ratio of 40 allows us to obtain monodisperse AuNPs 20-25 nm in diameter (FIG. 1).

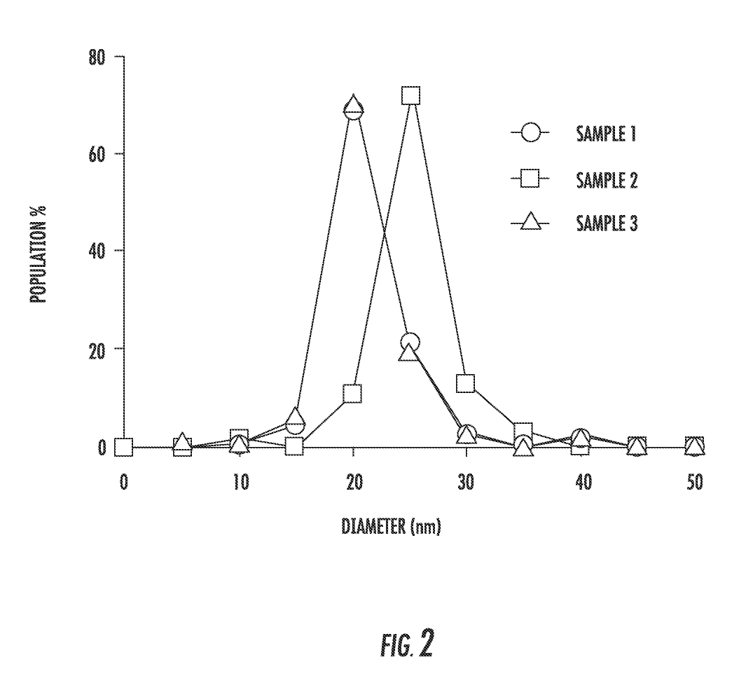

Batch to batch synthesis is consistent; FIG. 2 shows three independent batches with high monodispersity, which varied only by a few nanometers (FIG. 2).

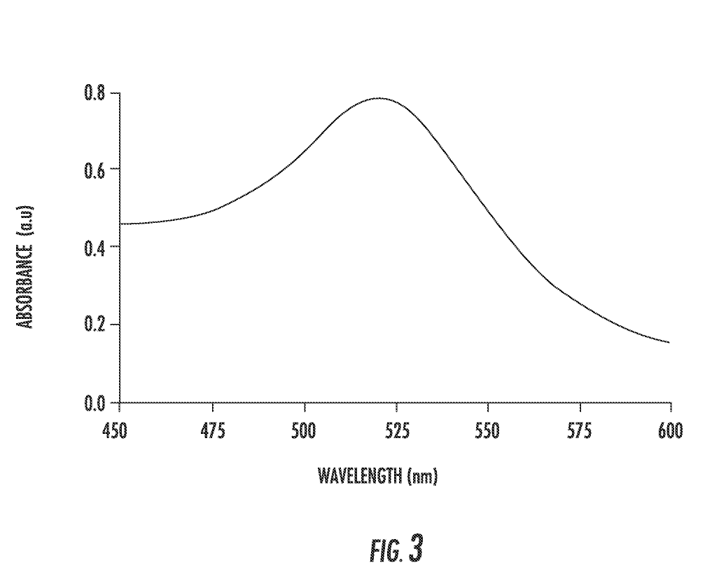

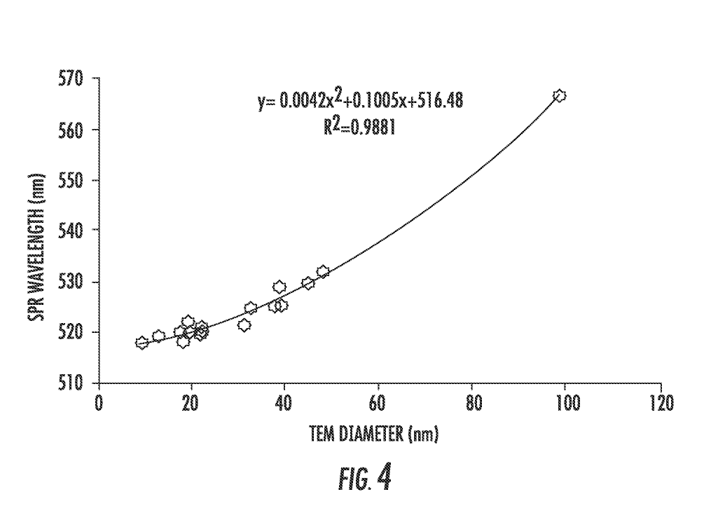

The surface plasmon resonance (SPR) wavelength at 520 nm indicates that there was no initial aggregation (FIG. 3). The 520 nm SPR wavelength also is yet another method to infer size is as expected (FIG. 4). UV-Vis measurements also are useful to investigate the aggregation of a sample. A decrease in absorbance over time is indicative of particles aggregating and falling out of solution. The citrate-stabilized AuNPs are very stable over the course of months with no change in the absorbance spectra due to their highly negative ZPs.

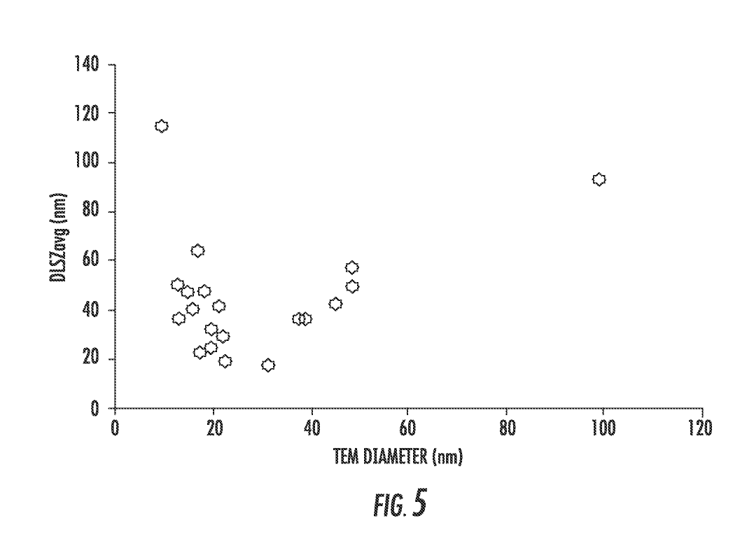

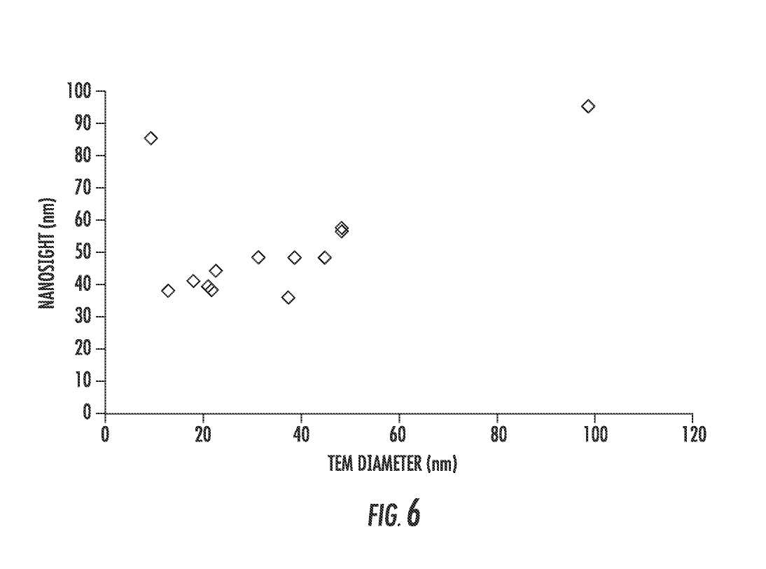

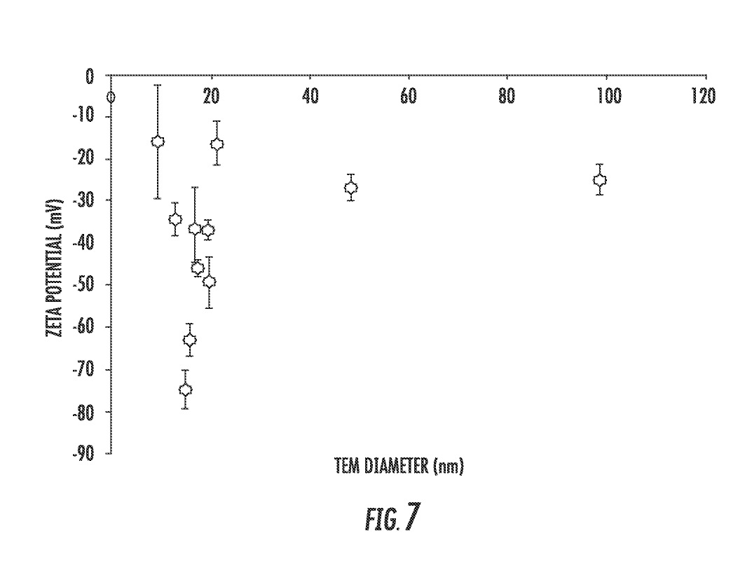

The AuNPs were characterized via TEM, DLS (FIG. 5), and NanoSight (FIG. 6). The ZP (by DLS) of the presently disclosed AuNPs is inversely proportional to size (FIG. 7). The AuNPs' ZP neutralizes as the concentration decreases; presumably due to citrate no longer being adsorbed to the AuNP surface (FIG. 8). It is important to layer the AuNPs at a concentration that allows the nanoparticles to not only be stable, but be capable of ionically complexing the first polymer layer.

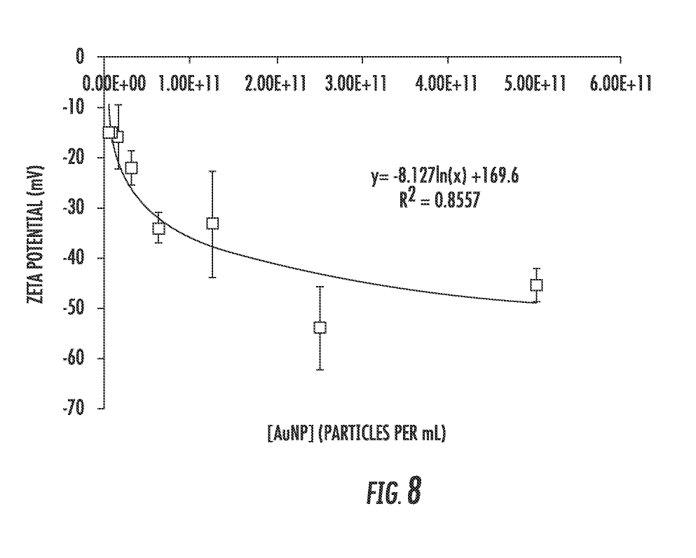

Citrate stabilized AuNPs directly post synthesis have a pH of the ultra pure distilled water as it is their solvent. As pH decreases, more of the citrate becomes uncharged and thus the ZP neutralizes (FIG. 9). As the ZP neutralizes the particles can approach one another with greater ease causing aggregation and instability as is shown with the red-shifting SPR wavelength, as well as the decreasing maximum absorbances. The maximum absorbances decrease as the concentration in solution appears to be less because large aggregates are falling out of solution.

### Polymer Synthesis

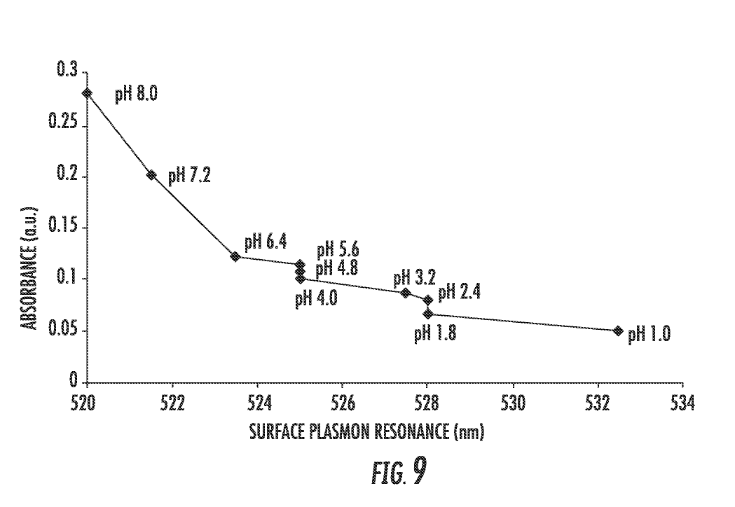

Diacrylates (BSS, B4) and amine side (S3, S4) chains were mixed and stirred on magnetic stir plate at 1000 RPMs at 90° C. (FIG. 10). Subsequently, amine-containing monomers (E6, E7) were used to end-cap the acrylate-terminated polymers at room temperature. Bhise N S, et al. Biomaterials, 2010, 31:31, 8088-96. The BSS-S3-E7 polymer is a disulfide bond-containing polymer, which is reduced by glutathione intracellularly and is not hydrolytically degraded, whereas the poly(beta aminoester) B4-S4-E6 is hydrolytically degraded. Because of B4-S4-E6's positive charge and buffering capacity it is able to escape the endosome by buffering. The buffering escape effect is known as the proton sponge effect. As the B4-S4-E6 buffers the endosome, more and more protons are shuttled into the endosome along with chloride ions and water due to charge neutralization and osmosis. As this occurs, the endosome lyses and the cargo (i.e., DNA and siRNA) can escape without being degraded.

### Layer-by-Layer (LbL) Technique

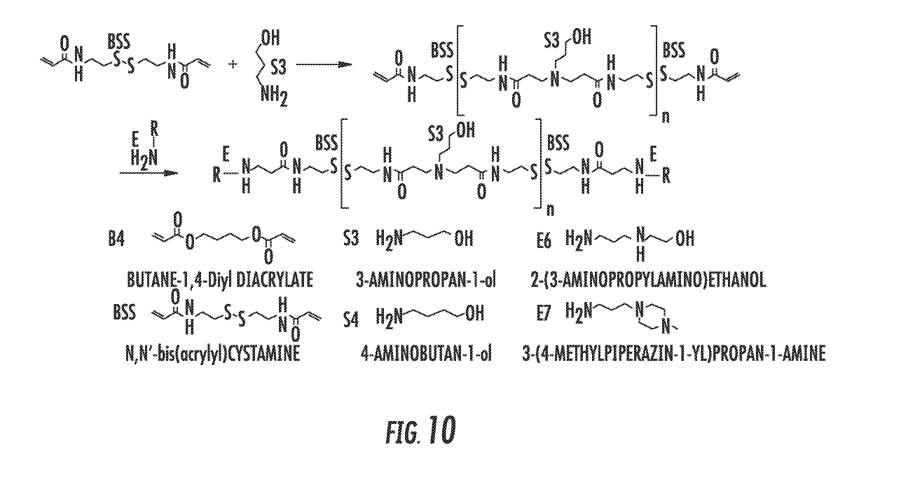

See generally, Lee S K, et al. Small, 2011, 7:3, 364-70; Elbakry, A, et al. Nano Lett., 2009, 9:5, 2059-64. In the presently disclosed methods, 58.3 μL of the AuNP solution at 1e11 particles per mL (absorbance of 0.145 a.u.) was placed in a microcentrifuge tube. 41.7 μL of BSS-S3-E7 at 5 mg/mL (rationale for choosing 5 mg/mL in FIGS. 11, 12 and 18I, which show the magnitude of the ZP to be the highest for the values tested, the size, as well as the highest cellular uptake, respectively) dissolved in 25 mM NaAc was subsequently added to the AuNP solution.

After 30 minutes of incubation, the solution is then centrifuged at 1.5 kref for 10 minutes. All of the supernatant is discarded. There is negligible product in the fluid at this point —even in siliconized microcentrifuge tubes. To recapture the product, 5 μL of dimethylsulfoxide (DMSO) is added to the vial and swirled around the edges. The DMSO turns a pinkish-red because it is pulling the ionic complexes off of the walls of the vial. It has been found that without DMSO, approximately only 15% of the AuNPs are preserved according to the absorbance maxima comparisons (concentration is linearly proportional to absorbance-Beer-Lambert's law). Furthermore, without DMSO, sonicating is necessary to resuspend the LbL AuNP complex, which damages the structural integrity of the nucleic acids, which will, in turn, decrease potential transfection capabilities.

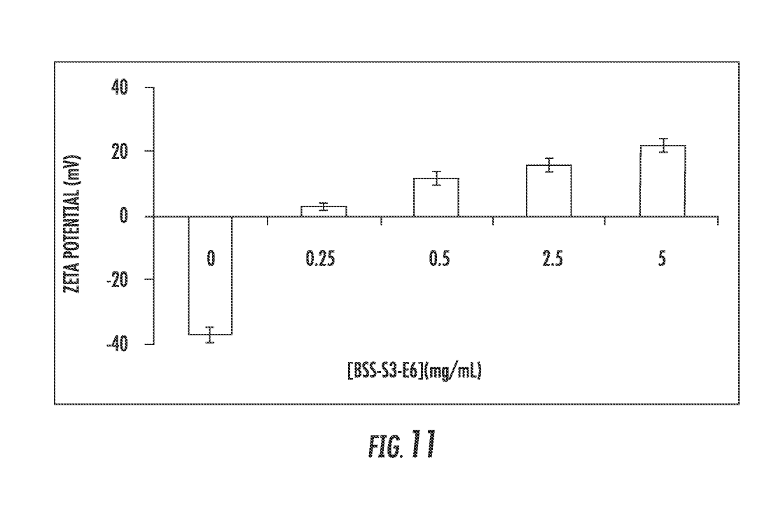

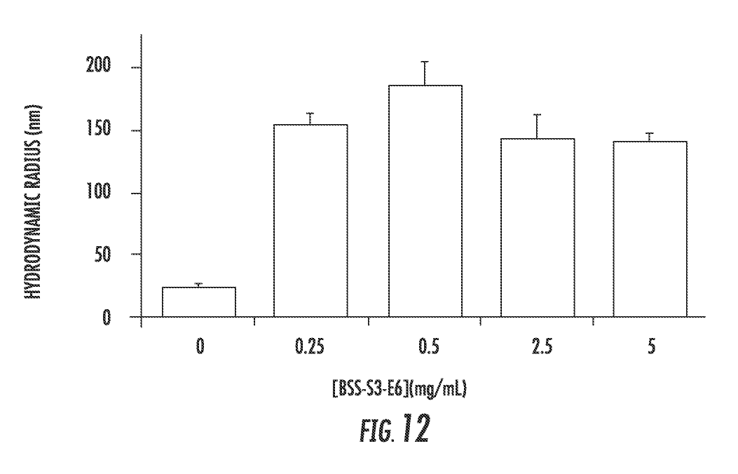

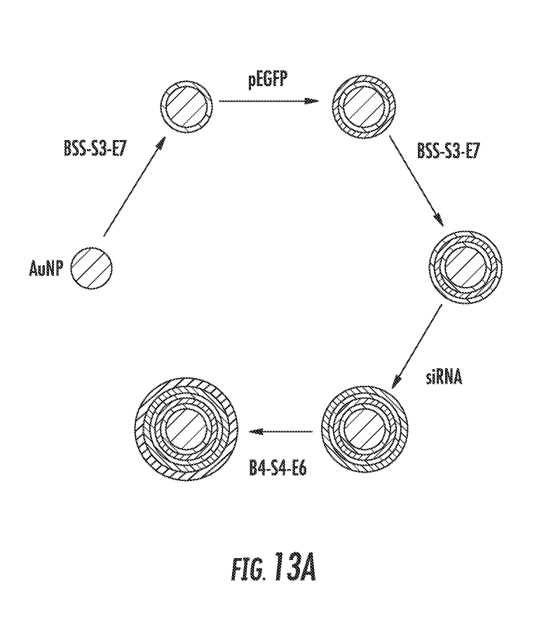

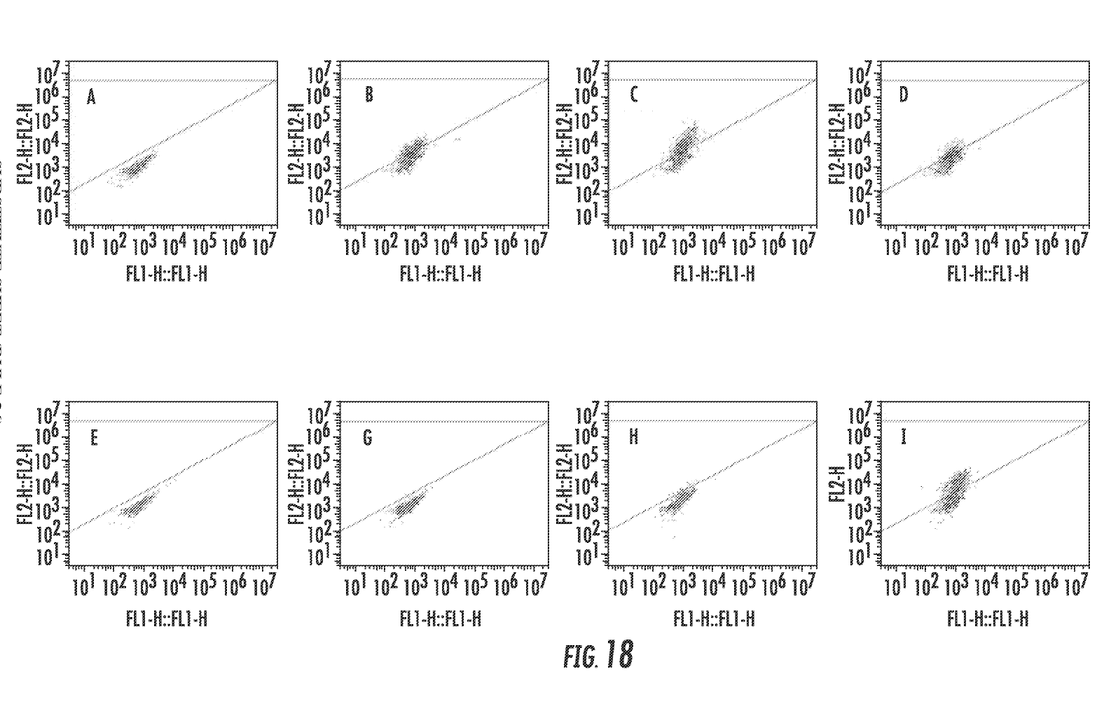

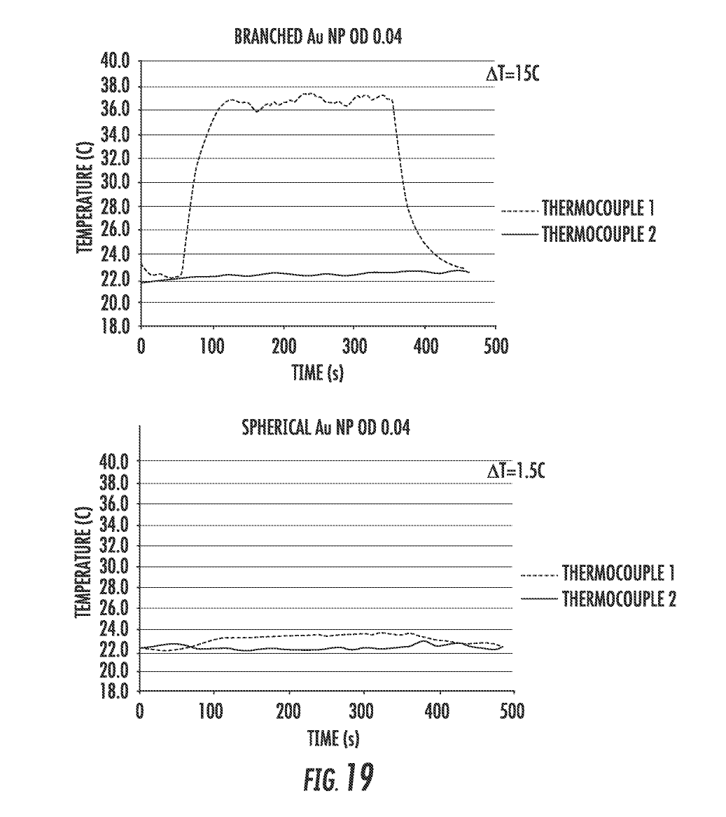

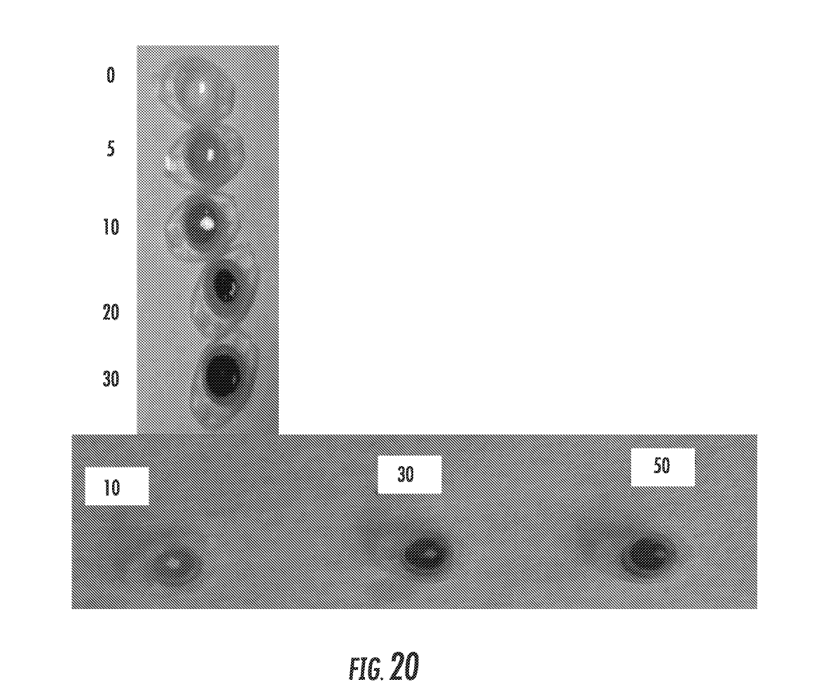

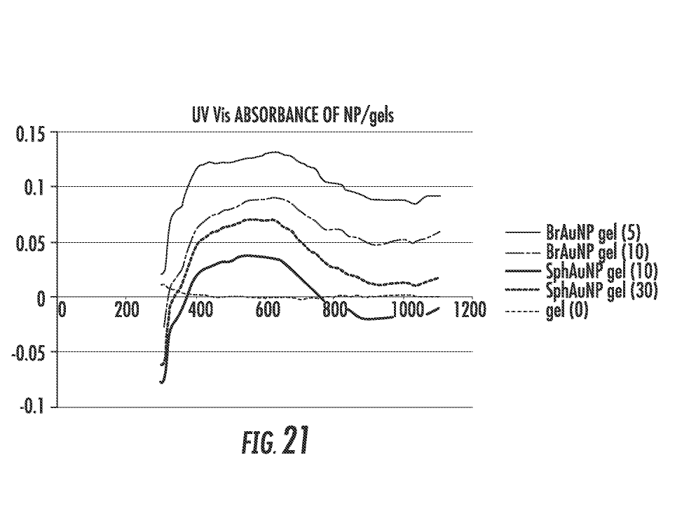

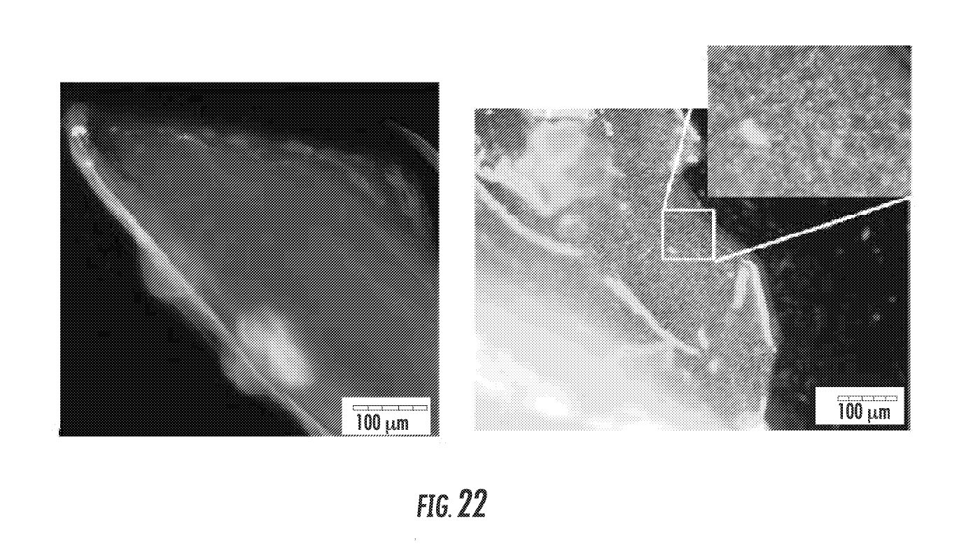

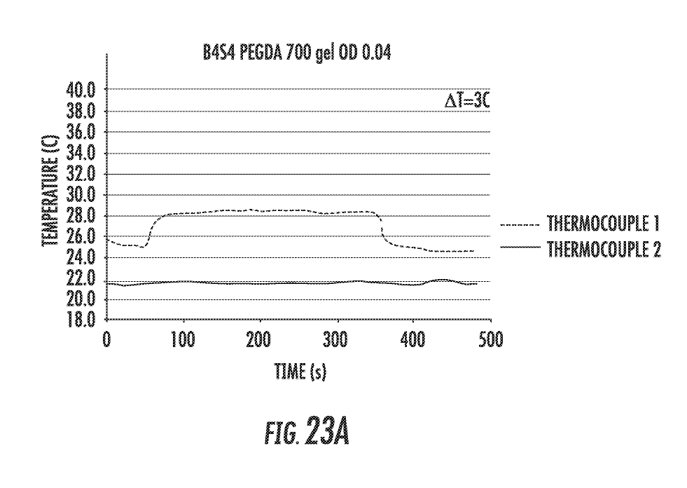

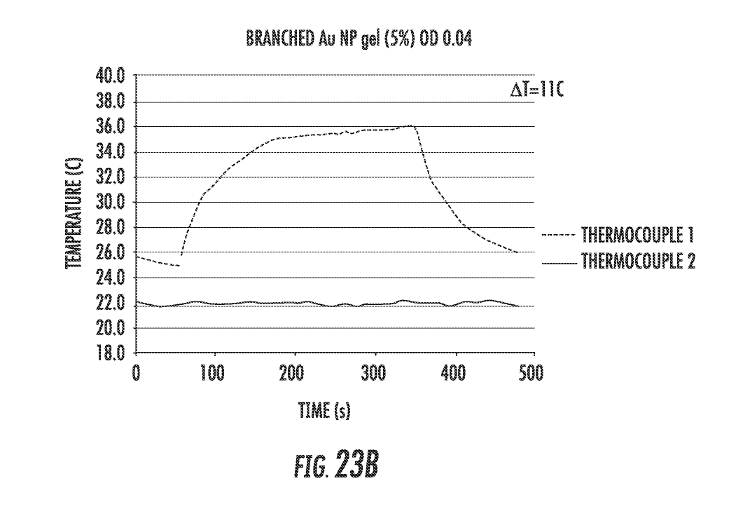

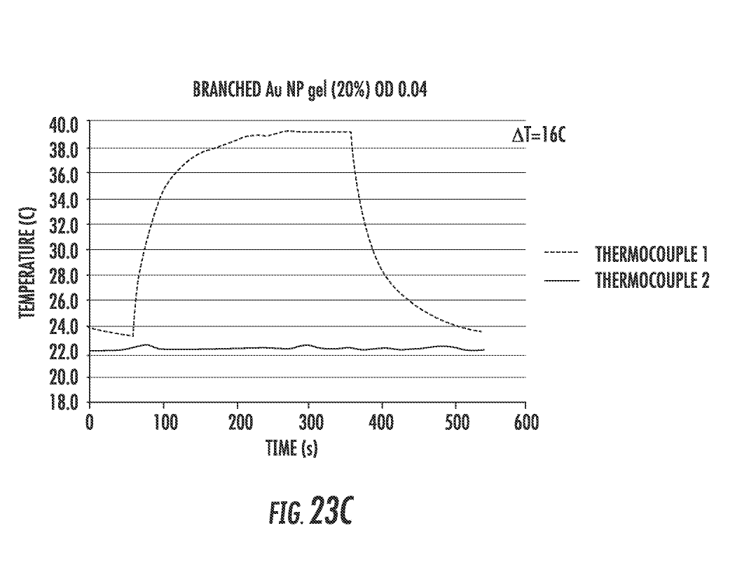

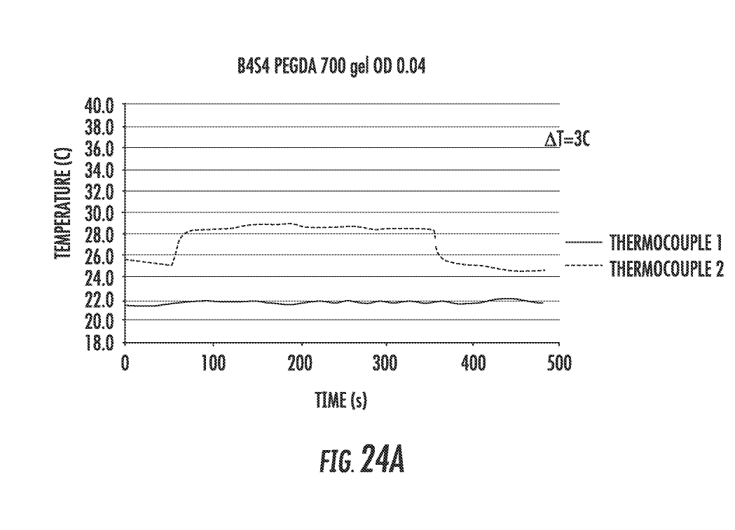

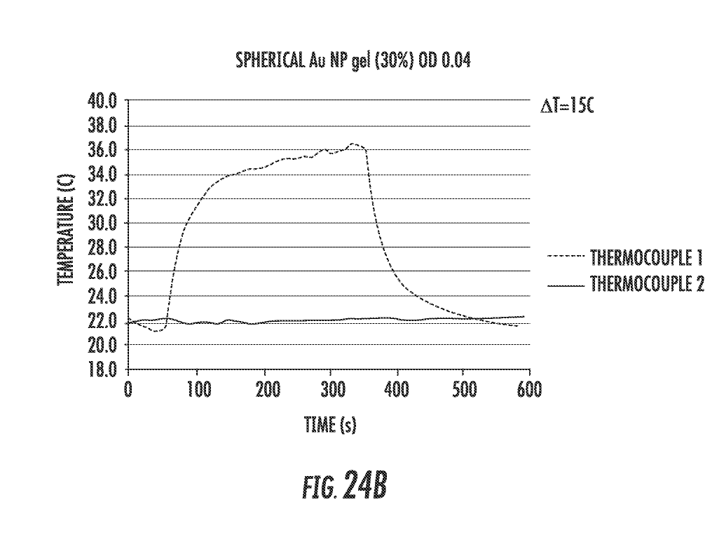

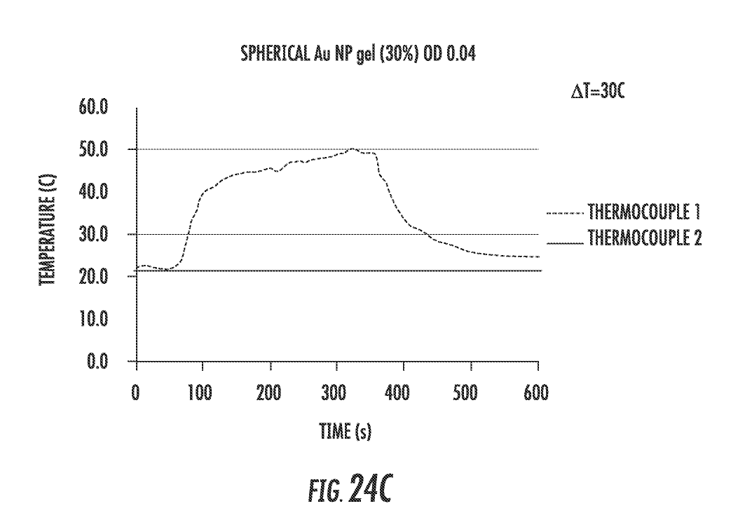

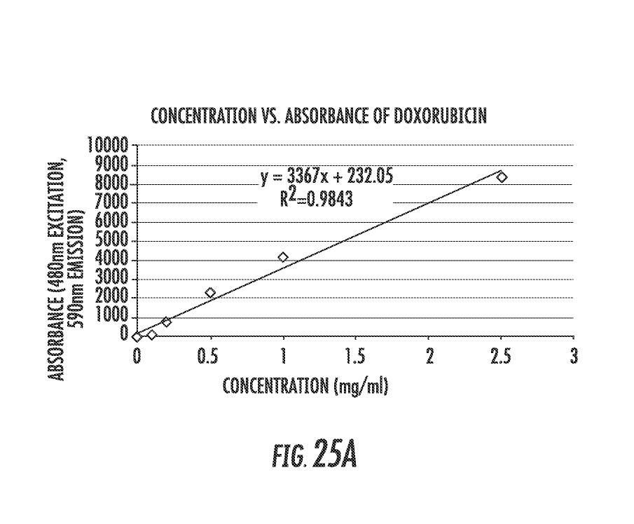

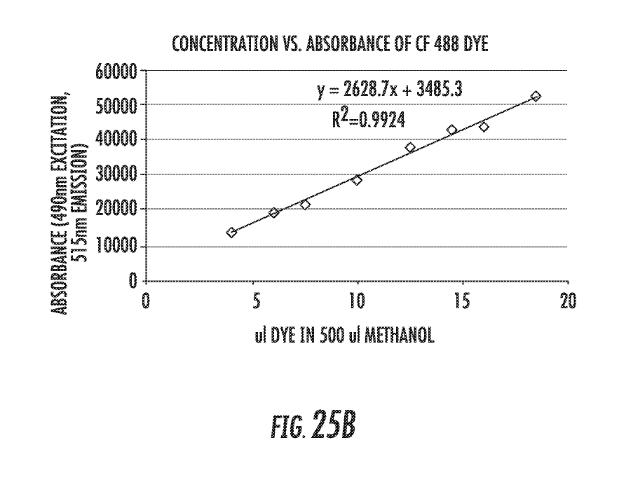

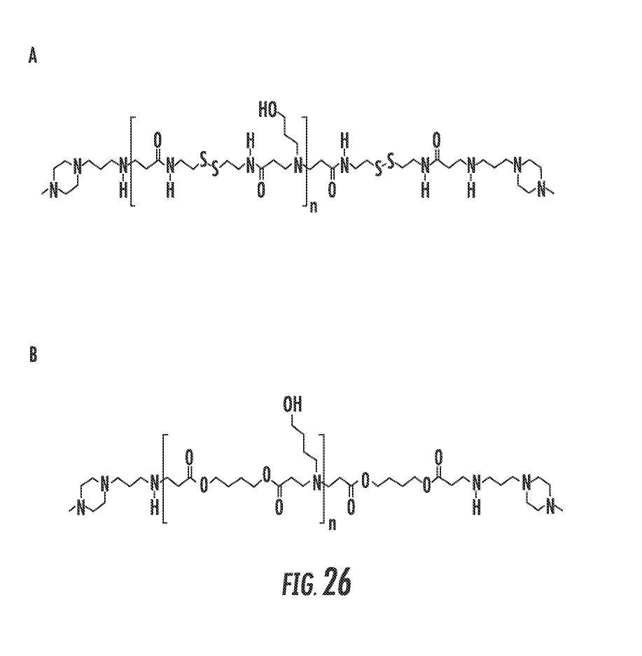

Subsequently the solution is filled again to 58.3 μL by adding 53.3 μL of 25 mM NaAc. The subsequent layers are then added in a similar fashion as was previously described (FIG. 13A). The LbL order is as follows: naked AuNP core, BSS-S3-E7 (5 mg/mL), DNA (0.5 mg/mL; rationale in FIG. 14, which shows the ZP's magnitude to be the highest for the values tested), BSS-S3-E7 (5 mg/mL), siRNA (4 μM), PBAE (B4-S4-E6 at 5 mg/mL; rationale in FIG. 18I, which shows the highest cellular uptake). After each layer is added and centrifuged all of the fluid contained in the vial is discarded and the product is resuspended using 5 μL of DMSO.

Referring now to FIG. 13B, a schematic of a representative layer-by-layer nanoparticle 3100 is provided. In some embodiments, nanoparticle 3100 comprises a nanoparticle core 3110: a first layer 3120 comprising a first cationic disulfide-reducible polymer, a second layer 3130 comprising a first anionic nucleic acid; a third layer 3140 comprising a second cationic disulfide-reducible polymer, wherein the first and the second cationic disulfide-reducible polymer can be the same or different; a fourth layer 3150 comprising a second anionic nucleic acid; and a fifth layer 3160 comprising a cationic hydrolytically degradable polymer.

In some embodiments, nanoparticle core 3110 comprises an inorganic nanoparticle core. In particular embodiments, the inorganic nanoparticle core comprises a gold nanoparticle core. In some embodiments, at least one of the first and second cationic disulfide-reducible polymer comprises a disulfide-containing poly-(amidoamine). In particular embodiments, the disulfide-containing poly(amidoamine) comprises BSS-S3-E7. In some embodiments, the cationic hydrolytically degradable polymer comprises a poly(β-aminoester). In particular embodiments, the poly(β-aminoester) comprises B4-S4-E6. One of ordinary skill in the art would recognize upon review of the presently disclosed subject matter that the disclosed layer-by-layer nanoparticles can comprises more alternating layers than depicted in FIG. 13B.

Further, ensuring the AuNPs' SPR does not shift significantly during the layering process is critical. A pH 5.2 buffer, namely NaAc at 25 mM, is a crucial solvent for the polymer and nucleic acids as the pH is not too low to cause significant aggregation of AuNPs, but low enough that it maintains the charge on the BSS-S3-E7 polymer and nucleic acids, allowing for the ionic complexation of the layers.

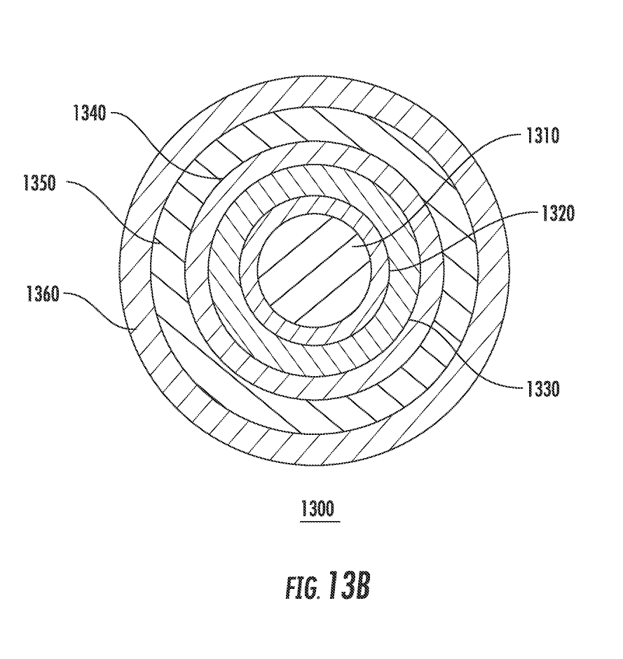

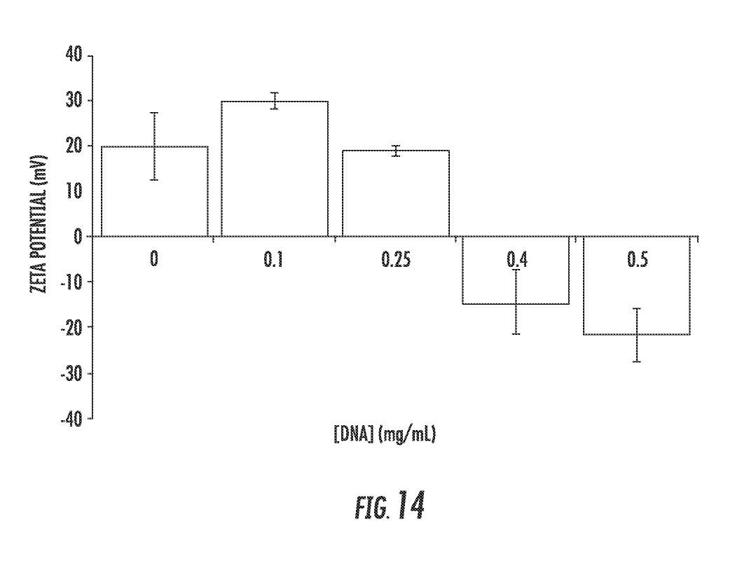

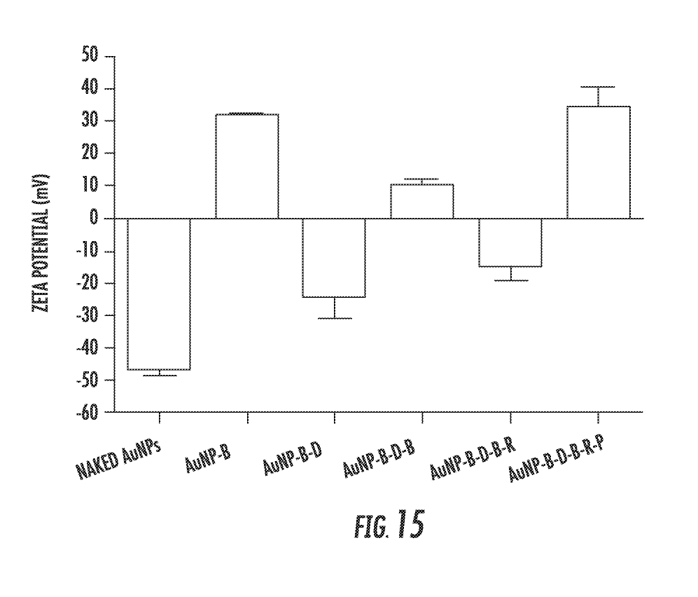

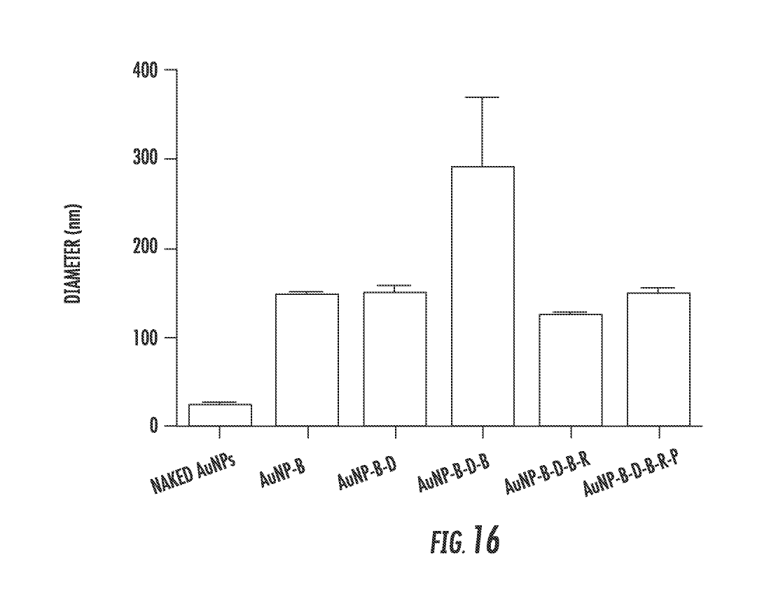

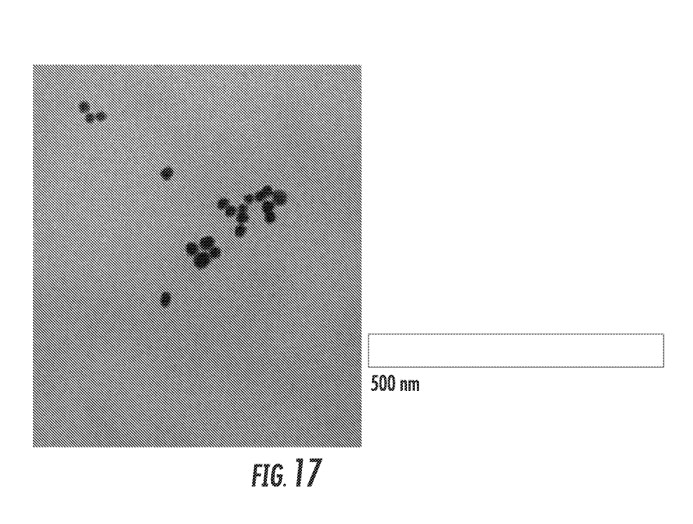

The zeta potential of the nanoparticles is reversed after each layer (FIG. 15), ranging from −46.04 to 34.04 mV. The naked AuNPs increased in size from 22.7±2.0 to 147.0±7.8 nm (via DLS) after the 5 layers were complexed. While the AuNPs' size after the first layer showed some aggregation, this aggregation did not significantly increase further with most subsequent layers including the last layer (FIGS. 16 and 17).

### Glioblastoma Uptake of LbL AuNPs

T-75 flasks of a gliobastoma cell lines (GB319) were grown to confluency. The GB319 cells were seeded at 5000 cells per well in a 96 well plate and allowed to culture for 24 hours to ensure the cells were adhered to the flask. After 24 hours of incubation, the cells were transfected with either nothing (FIG. 18A), Lipofectamine 2000 (gene delivery gold standard of scientific community) (FIG. 18B), PBAE polyplex at a polymer:DNA weight ratio of 60 (FIG. 18C), the AuNP LbL system ending in poly(ethylene imine) rather than PBAE (FIG. 18D), and the AuNP LbL system ending in either 0, 0.5, 2 or 5 mg/mL of PBAE polymer B4-S4-E6 (FIG. 18E-I, respectively).

In summary, siRNA and DNA can be simultaneously ionically complexed to AuNPs for co-delivery using two cationic polymers with unique degradable mechanisms. BSS-S3-E7 is a disulfide-containing-poly(amidoamine) which can be reduced and degraded upon uptake into the cell with increased glutathione levels. B4-S4-E6 is a poly(β-aminoester) which is degraded by hydrolysis. The two uniquely degrading polymers allow us to cater the release kinetics of DNA and siRNA by varying the order and the number of the layers of the polymers. Furthermore, varying the disulfide density within the poly(amido amine) polymer will allow further control over the release kinetics of the DNA and siRNA.

The LbL layer ending in PBAE appears superior to polyethylenimine (PEI) and Lipofectamine 2000 and seems comparable to PBAE polyplexes in endosomal uptake in the glioblastoma cell line used according to the flow cytometry uptake data. Without the PBAE on the outer coating the LbL AuNPs are not uptaken into the cells. Increasing LbL PBAE concentrations increases uptake which will likely lead to enhanced transfection.

Accordingly, the presently disclosed subject matter provides a theranostic technology that can deliver combinations of genetic therapies along with an agent for imaging and potential photothermal therapy.

### Example 2

### Thermo-Sensitive Gels with Heatable Nanoparticles for Dual Hyperthermia and Drug Delivery Systems Methods

### Gel Synthesis

Gels were synthesized using the following:

Base Polymer:

B4-S5, B5-S5 (both 1.2:1);

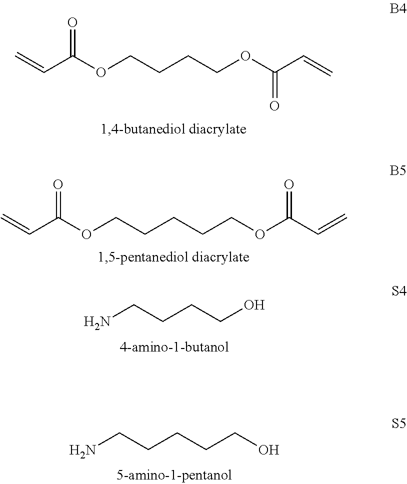

Acrylate Crosslinkers:

### PEGDA 258 Dalton MW; PEGDA 700 Dalton MW:

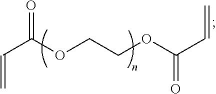

Trimethylolpropane triacrylate:

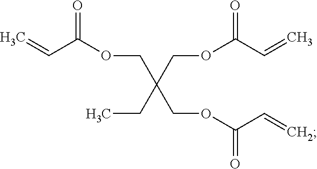 [SMILES: C=CC(=O)OCC(CC)(COC(=O)C=C)COC(=O)C=C]

and 1,4-butanediol diacrylate:

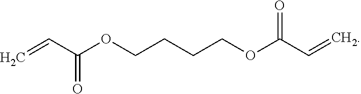

Photoinitiator: Irgacure 2959. Ratios of 10:10, 10:20, 10:30, 10:40, 0:20 (0.05% Irgacure)

### Gel/Nanoparticle Synthesis

The IMEC procedure for Au and FeCoO nanoparticles. Homogeneous distribution of nanoparticles was demonstrated throughout the gel.

### Drug Release

Measure gels for drug retention at 37° C. and release at 45° C.

### Cells

Demonstrate system on MDA-231 cells using MTT assay.

### Example 3

A Layer-by-Layer Approach to Co-Deliver DNA and siRNA Via AuNPs: A Potential Platform for Modifying Release Kinetics

Many genetic disorders could be substantially mitigated or cured by gene therapy. To date there are no FDA-approved gene therapies due to inadequacies of safety and efficacy. Viral vectors transduce well but are immunogenic, whereas polymeric vectors are relatively safer but lack efficacy. Innovative nucleic acid vectors capable of improving transfection efficacy and control of nucleic acid delivery kinetics would substantially benefit technology translation from bench-top to clinic. Inorganic gold nanoparticles (AuNP) are a promising candidate as a nucleic acid delivery platform, as they are monodisperse, biocompatible, readily surface modifiable, and have unique optical properties (Sunshine et al., 2011).

In some embodiments, thiolated carboxylic acid was added to citrate-stabilized AuNPs (MAuNPs). The LbL process was used in 150-mM sodium acetate (Lee et al., 2011; Elbakry et al., 2009). The size of the mAuNPs was analyzed via TEM and nanoparticle tracking analysis. The zeta potential was measured via DLS. A cell titer assay was used to measure metabolic activity. Flow cytometry was used to determine efficacy (hGBM cells).

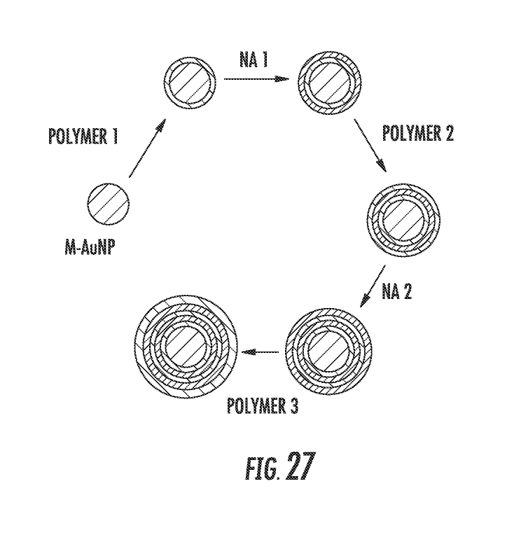

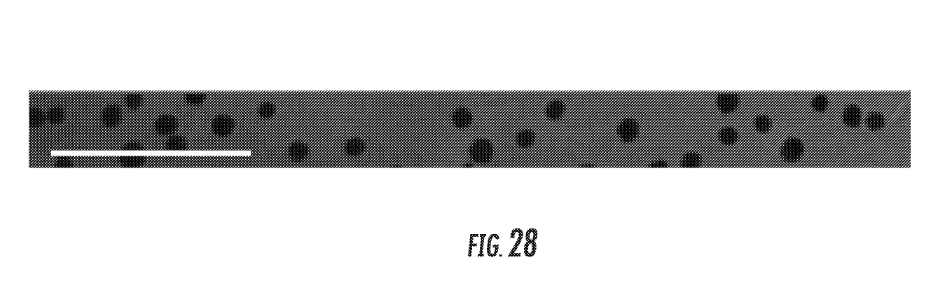

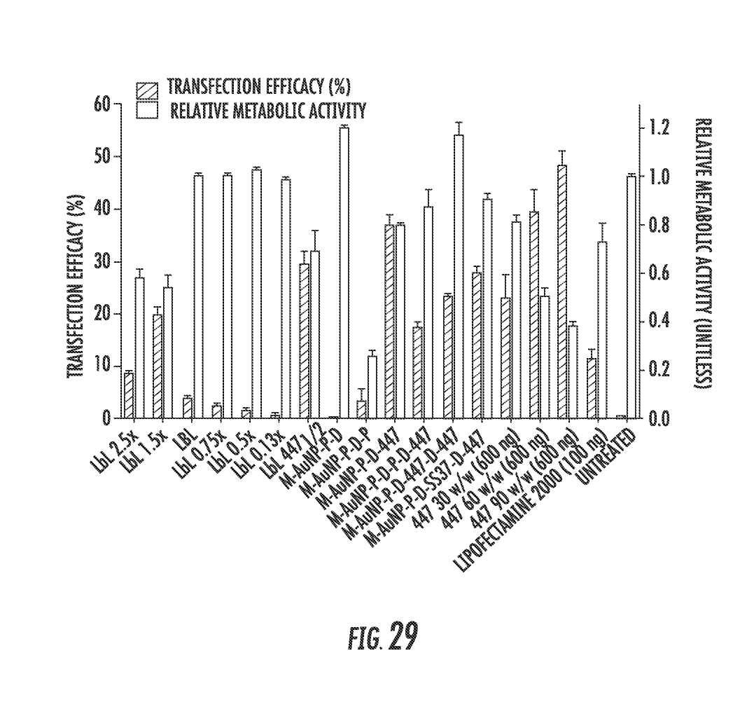

FIG. 26 shows the structures of a representative disulfide-reducible poly(amidoamine), BSS-S3-E7 (Lin et al., 2007) and a representative hydrolytically degradable poly(β-aminoester), B4-S4-E7 (Bhise et al., 2010). FIG. 27 shows a schematic of the process by which M-AuNPs are coated by polymer and nucleic acid (NA) layers. Referring once again to FIG. 27, in some embodiments, a gold nanoparticle is coated with a first polymer (e.g., Polymer 1), then coated with a first nucleic acid (e.g., NA 1), then coated with a second polymer (e.g., Polymer 2), then coated with a second nucleic acid (e.g., NA 2), and finally coated with a third polymer (e.g., Polymer 3). In representative embodiments, Polymer 1 comprises PET, NA 1 comprises DNA, Polymer 2 comprises BSS-S3-E7, NA 1 comprises DNA, and Polymer 3 comprises B4-S4-E7. A TEM of monodisperse, 15-nm citrate-stabilized AuNPs is shown in FIG. 28.

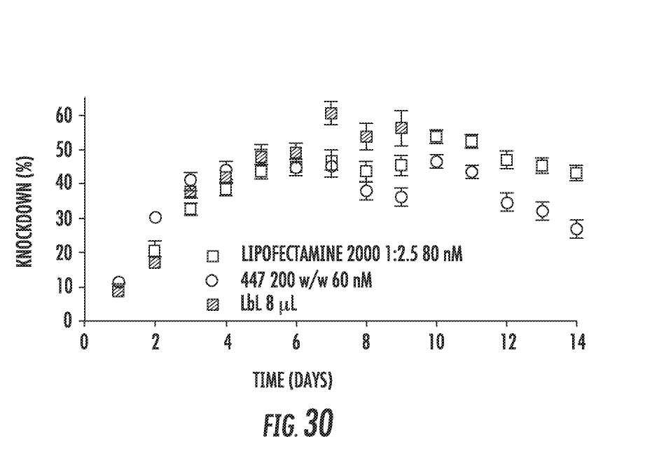

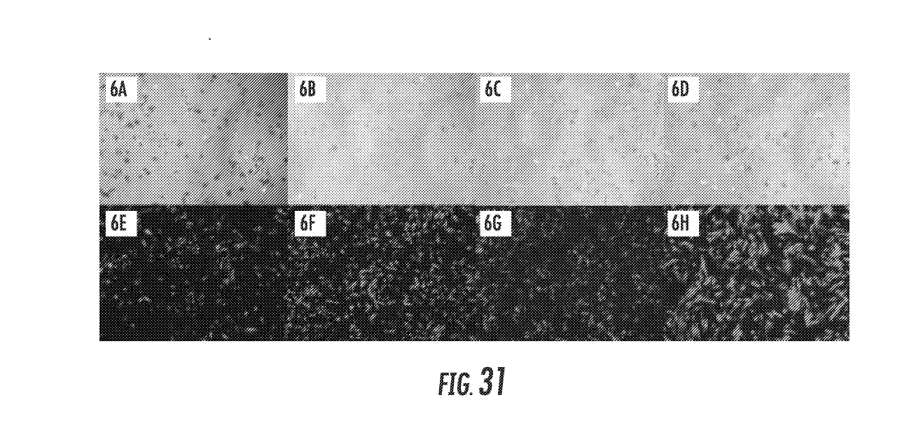

FIG. 29 shows the transfection efficacy and relative metabolic activity of various formulations (P is PEI, D is DNA, 447 and SS37 are the B4-S4-E7 and BSS-S3-E7 polymers, respectively. LbL is MAuNP-P-D-SS37-siRNA-447). FIG. 30 shows the knockdown in time of the LbL, Lipofectamine and 447 formulations.

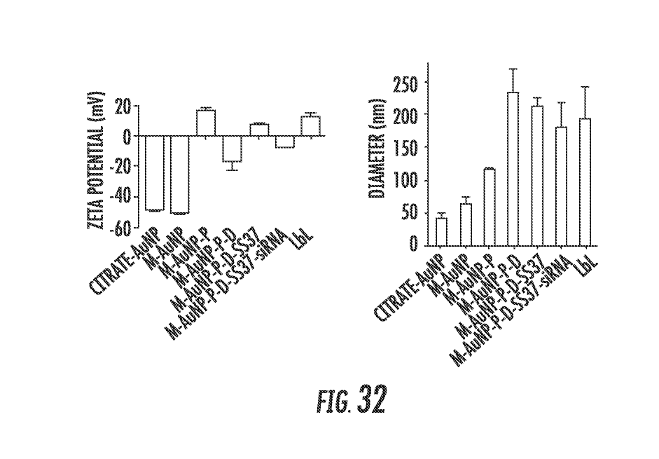

FIG. 31 shows dsRed expression at day 2 (6A-6D): (6A) LbL 1.5× dose, (6B) LbL, (6C) Lipofectamine, (6D) 447; eGFP knockdown at day 9 (6E-6H): (6E) LbL eGFP siRNA, (6F) LbL scr-siRNA, (6G) Lipofectamine eGFP siRNA, (6H) Lipofectamine scr-siRNA. LbL particles maintained 100% viability and resulted in 4% dsRed expression by day 2 and 50% knockdown of GFP at day 9. FIG. 32 shows the reversal of zeta potential after each successive layer (left) and diameter of each of the layers (right) after two washings using the LbL formulation.

Accordingly, the presently disclosed subject matter provides a layer-by-layer (LbL) system, which alternately ionically complexes anionic AuNPs to two unique cationic polymers and two anionic nucleic acids. The siRNA and DNA can be ionically complexed to AuNPs for co-delivery while maintaining functionality using two cationic polymers with unique degradable mechanisms. The use of polymer 447, i.e., B4-S4-E7, as the last layer was found to be superior to PEI or no polymer. As AuNPs were layered, size rapidly increased which is indicative of multiple AuNP cores present. By altering the number, order and degradability of the polymer layers, the expression and knockdown could potentially be controlled kinetically.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Sunshine, et al. Therap. Delivery, 2011, 2(4), 493-521;
Bhise N S, et al. Biomaterials, 2010, 31:31, 8088-96;
Lee S K, et al. Small, 2011, 7:3, 364-70;
Elbakry, A, et al. Nano Lett., 2009, 9:5, 2059-64.
D. Putnam, Polymers for gene delivery across length scales, Nature Materials, vol. 5, pp. 439-51, June 2006;
E. Check, Gene therapy put on hold as third child develops cancer, Nature, vol. 433, pp. 561-561, FEB 10 2005; and
O. Boussif, F. Lezoualc'h, M. A. Zanta, M. D. Mergny, D. Scherman, B. Demeneix, and J. P. Behr, A versatile vector for gene and oligonucleotide transfer into cells in culture and in vivo: polyethylenimine, Proc Natl Acad Sci USA, vol. 92, pp. 7297-301, Aug. 1 1995.
Lin, C. et al. Bioconjugate Chem., 2007, 18, 138-45.

Although the foregoing subject matter has been described in some detail by way of illustration and example for purposes of clarity of understanding, it will be understood by those skilled in the art that certain changes and modifications can be practiced within the scope of the appended claims.

1. A composite comprising a polymeric network or gel and an inorganic nanoparticle, wherein the inorganic nanoparticle can generate heat upon external stimulation.

2. The composite of claim 1, wherein the polymeric network or gel comprises a degradable polymer.

3. The composite of claim 1, wherein the polymeric network or gel comprises a compound synthesized by the following method including one or more of the following monomers and combinations thereof:

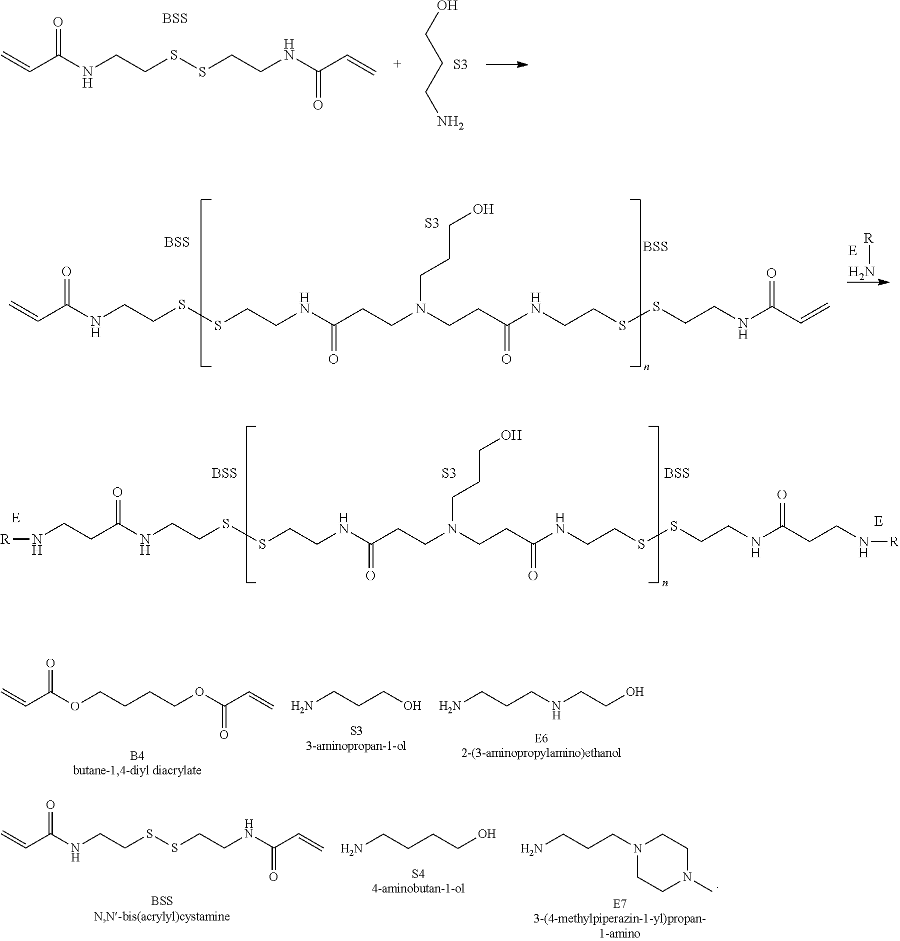

4. The composite of claim 1, wherein the polymeric network or gel comprises one or more backbones and side chains selected from the following monomers:

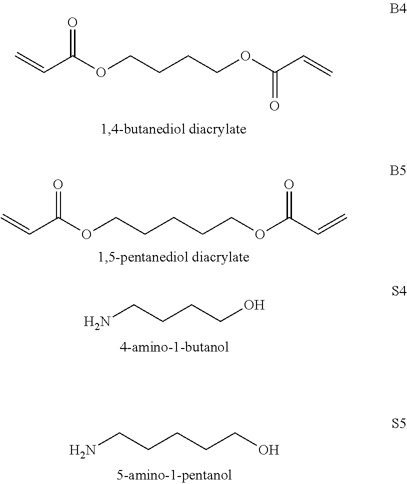

5. The composite of claim 1, wherein the polymeric network or gel further comprises polyethylenimine (PEI).

6. The composite of claim 1, wherein the inorganic nanoparticle comprises a gold nanoparticle.

7. The composite of claim 6, wherein the gold nanoparticle can be activated when exposed to a particular wavelength of light.

8. The composite of claim 1, wherein the inorganic nanoparticle comprises a magnetically-activated nanoparticle.

9. The composite of claim 1, further comprising a cargo.

10. The composite of claim 9, wherein the cargo is selected from the group consisting of a therapeutic agent, a biosensor, and a biological molecule.

11. The composite of claim 10, wherein the therapeutic agent is selected from the group consisting of a gene, DNA, RNA, siRNA, miRNA, isRNA, agRNA, smRNA, a nucleic acid, a peptide, a protein, a chemotherapeutic agent, a hydrophobic drug, a small molecule drug, and combinations thereof.

12. The composite of claim 10, wherein the therapeutic agent can be released from the composite in response to a change in temperature of composite.

13. An implant comprising a composite of claim 1.

14. The implant of claim 13, wherein the implant is suitable for on-demand or extended release delivery of a therapeutic agent to a subject.

15. The composite of claim 1, wherein the particle has a size of about 20 nm to about 100 nm.

16. The composite of claim 1, wherein the particle has a size of about 100 nm to about 300 nm.

17. The composite of claim 1, wherein the particle has a size of about 300 nm to about 1000 nm.

18. The composite of claim 1, wherein the particle has a size of about 1 micron to about 10 microns.

19. The composite of claim 1, wherein the particle has a size of about 10 microns to about 30 microns.

20. A composite comprising a core inorganic nanoparticle and one or more layers or coatings of a plyelectrolyte.

21. The composite of claim 20, wherein the one or more layers or coatings of a polyelectrolyte comprises one or more layers or coatings of materials which alternate in charge between positive and negative.

22. The composite of claim 20, wherein the one or more layers or coatings comprise a charged biological molecule.

23. The composite of claim 20, wherein the polyelectrolyte comprises a degradable polymer.

24. The composite of claim 20, wherein the polyelectrolyte comprises a compound synthesized by the following method including one or more of the following monomers and combinations thereof:

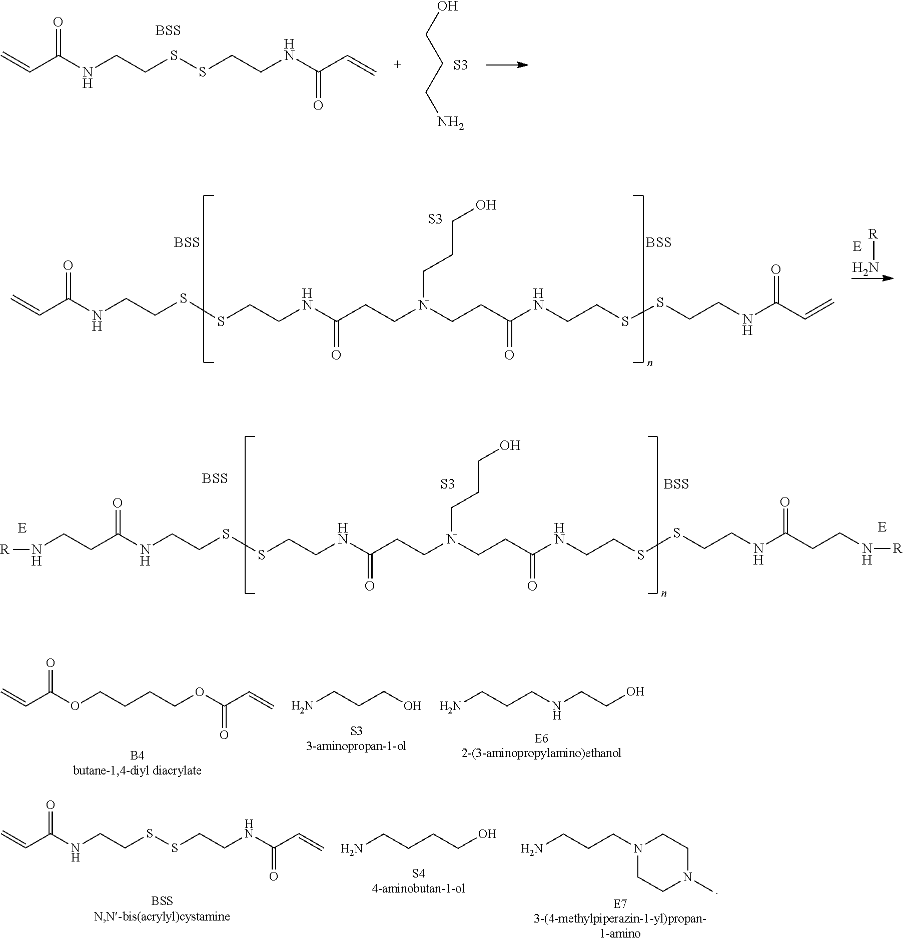

25. The composite of claim 20, wherein the polyelectrolyte comprises one or more backbones and side chains selected from the following monomers:

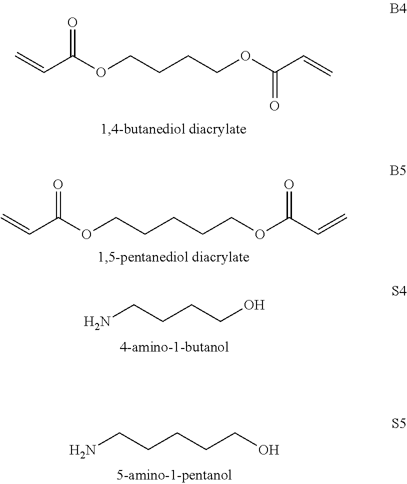

26. The composite of claim 20, wherein the one or more layers or coatings of a polyelectrolyte polymeric network or gel comprise polyethylenimine (PEI).

27. The composite of claim 20, wherein the inorganic nanoparticle comprises a gold nanoparticle.

28. The composite of claim 27, wherein the gold nanoparticle can be activated when exposed to a particular wavelength of light.

29. The composite of claim 20, wherein the inorganic nanoparticle comprises a magnetically-activated nanoparticle.

30. The composite of claim 20, further comprising a cargo.

31. The composite of claim 30, wherein the cargo is selected from the group consisting of a therapeutic agent, a biosensor, and a biological molecule.

32. The composite of claim 31, wherein the therapeutic agent is selected from the group consisting of a gene, DNA, RNA, siRNA, miRNA, isRNA, agRNA, smRNA, a nucleic acid, a peptide, a protein, a chemotherapeutic agent, a hydrophobic drug, a small molecule drug, and combinations thereof.

33. The composite of claim 31, wherein the therapeutic agent can be released from the composite in response to a change in temperature of the composite.

34. An implant comprising a composite of claim 20.

35. The implant of claim 34, wherein the implant is suitable for on-demand or extended release delivery of a therapeutic agent to a subject.

36. A sensor comprising a composite of claim 20.

37. The composite of claim 20, wherein the particle has a size of about 20 nm to about 100 nm.

38. The composite of claim 20, wherein the particle has a size of about 100 nm to about 300 nm.

39. The composite of claim 20, wherein the particle has a size of about 300 nm to about 1000 nm.

40. The composite of claim 20, wherein the particle has a size of about 1 micron to about 10 microns.

41. The composite of claim 20, wherein the particle has a size of about 10 microns to about 30 microns.

42. A nanoparticle comprising:
a nanoparticle core;
a first layer comprising a first cationic polymer;
a second layer comprising a first anionic nucleic acid;
a third layer comprising a second cationic polymer, wherein the first and the second cationic polymer can be the same or different;
a fourth layer comprising a second anionic nucleic acid, wherein the first anionic and the second anionic nucleic acid can be the same or different; and
a fifth layer comprising a third cationic degradable polymer.

43. The nanoparticle of claim 42, wherein the nanoparticle core comprises an inorganic nanoparticle core.

44. The nanoparticle of claim 43, wherein the inorganic nanoparticle core comprises a gold nanoparticle core.

45. The nanoparticle of claim 42, wherein the first cationic polymer comprises polyethylenimine (PEI).

46. The nanoparticle of claim 42, wherein the second cationic polymer comprises a disulfide-reducible poly(amidoamine).

47. The nanoparticle of claim 46, wherein the disulfide-reducible poly(amidoamine) comprises BSS-S3-E7.

48. The nanoparticle of claim 42, wherein the third cationic degradable polymer comprises a hydrolytically degradable polymer.

49. The nanoparticle of claim 48, wherein the hydrolytically degradable polymer comprises a poly(β-aminoester).

50. The nanoparticle of claim 49, wherein the poly(β-aminoester) comprises B4-S4-E7.

51. The nanoparticle of claim 42, wherein the first anionic and the second anionic nucleic acid are selected from the group consisting of DNA and siRNA.
